(19) United States
(12) Patent Application Publication
(10) Pub. No.: US 2019/0127687 A1
(43) Pub. Date: May 2, 2019

(54) METHODS OF EXPANDING HUMAN PREPUBERTAL SPERMATOGONIAL STEM CELLS

(71) Applicant: NAM TRAN, San Francisco, CA (US)

(72) Inventors: NAM TRAN, San Francisco, CA (US); JAMES SMITH, San Francisco, CA (US)

(21) Appl. No.: 15/395,704

(22) PCT Filed: Jul. 2, 2015
(86) PCT No.: PCT/US15/39094

§ 371 Date: Dec. 30, 2016

Related U.S. Application Data
(60) Provisional application No. 62/020,773, filed on Jul. 3, 2014.

(51) Int. Cl. C12N 5/076; A61K 35/52; A61P 15/08

(52) U.S. Cl. CPC C12N 5/061; C12N 2502/246; A61P 15/08; A61K 35/52

(57) ABSTRACT

The invention comprises methods of identifying and isolating spermatagonial stem cells in testicular tissue samples by the use of markers unique to spermatagonial stem cells. The invention defines and characterizes a unique testicular cellular niche wherein spermatagonial stem cells reside. The invention also comprises methods of identifying and isolating multipotent testicular stromal cells, on which spermatagonial stem cells can be cultured. Cultured spermatagonial stem cells, derived from prepubertal males or cancer patients can be utilized to generate viable spermatozoon or may be subsequently transplanted back into the donor to restore fertility.

### CROSS-REFERENCE TO RELATED APPLICATIONS

This application claims the benefit of priority to: U.S. Provisional Application Ser. No. 62/020,773, entitled “Methods of Expanding Human Prepubertal Spermatogonial Stem Cells,” filed Jul. 3, 2014, the contents which are hereby incorporated by reference.

Statement Regarding Federally Funded Research or Development: Not Applicable.

### BACKGROUND OF THE INVENTION

Approximately 7,500 boys are diagnosed with cancers annually in the United States. Although patients have seen significant survival improvements with treatments, many suffer from permanent sterilization as a result of cancer treatments. For post-pubertal boys and adults, fertility preservation with cryopreserved semen is highly successful. Unfortunately, this option is not feasible for pre-pubertal boys. Pre-pubertal boys treated with high-dose chemotherapy and other regimens that cause sterility do not have an established means of fertility preservation as no established in vitro technique exists to isolate, expand, and mature purified spermatogonial stem cells (SSCs) to functional sperm in humans. Accordingly, there is a need in the art for methods of identifying, isolating, culturing, and expanding purified SSC's in humans.

### SUMMARY OF THE INVENTION

The present invention, in various embodiments, provides the art with methods for the identification, isolation, culture, expansion, maturation, and transplantation of SSC's in humans. The invention further encompasses methods for the diagnosis and treatment of infertility in general. The invention further encompasses compositions of matter derived by or associated with the methods disclosed herein.

In one aspect, the invention comprises methods of identifying SSC's in testicular tissue samples by the use of markers unique to SSC's. The invention further includes methods of isolating SSC's from a sample by means of the unique SSC markers. In another aspect, the invention defines and characterizes a unique testicular cellular niche wherein SSC's reside. In another aspect, the invention comprises methods of identifying multipotent testicular stromal (“MTS”) cells which reside in the SSC niche, using unique markers. In another aspect, the invention comprises methods of isolating and culturing MTS cells.

In another aspect, the invention comprises methods of culturing SSC's using MTS cells. In another aspect, the invention comprises methods of utilizing cultured SSC's to generate viable spermatozoon. In yet another aspect, the invention comprises the transplantation of SSC's into infertile individuals to restore fertility. In one embodiment, the testicular tissue sample from which SSC's are isolated is obtained from a cancer patient. In one embodiment, the testicular tissue sample from which SSC's are isolated is a prepubertal male.

In one aspect, the invention encompasses the identification of SSC genes involved in male fertility. In another aspect, the invention comprises methods of diagnosing infertility by use of the highly conserved male fertility genes disclosed herein. In another aspect, the invention comprises methods of treating male infertility by gene therapy to restore normal forms or expression patterns of highly conserved SSC genes which are mutated or being aberrantly expressed.

These various aspects and embodiments of the invention will next be described in detail.

### BRIEF DESCRIPTION OF THE DRAWINGS

FIG. 1. FIG. 1 depicts expression patterns in THY1+, SSEA-4+, and THY1−/SSEA-4− cells. THY1+ cells expressed high levels of VIM, but lack DAZL, VASA, DMC1, SYCP3, PRM2, and ACR. SSEA-4+ cells expressed high levels of DAZL and VASA with minimal expression of VIM and meiotic or spermatid markers. THY1−/SSEA-4− cells expressed high levels DAZL, VASA, DMC1, SYCP3, PRM2, and ACR. Expression levels between groups were significantly different for all genes examined. p<0.05 by ANOVA.

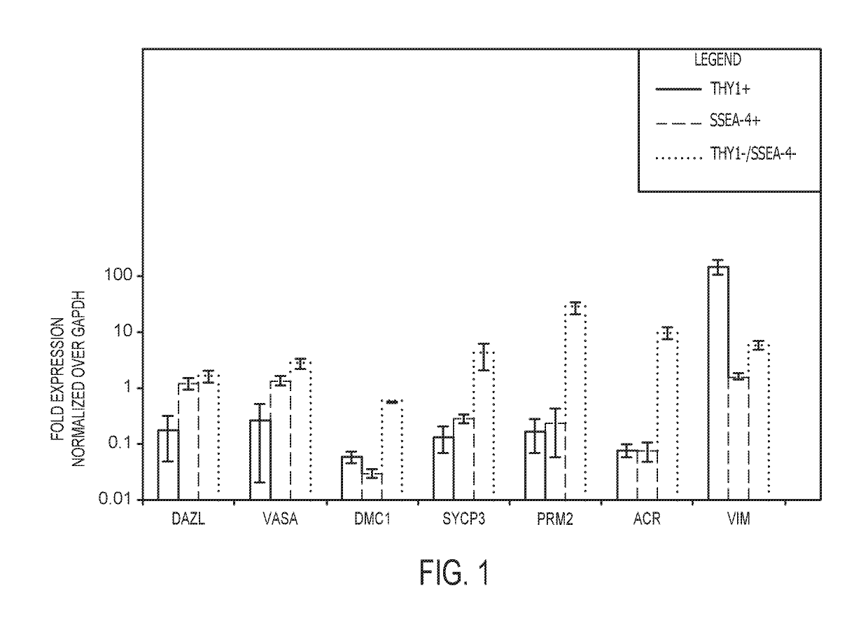

FIG. 2. depicts differential expression of ZBTB16, GFRA1, and GPR125 between THY1+ and SSEA-4+ populations. p<0.05 by Student t-test.

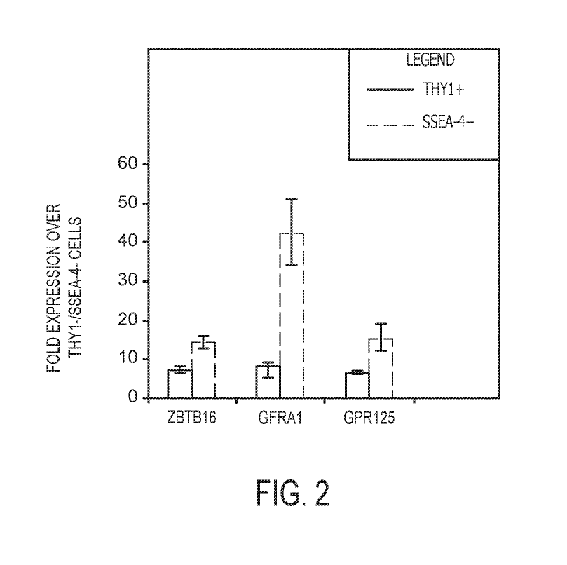

### DETAILED DESCRIPTION OF THE INVENTION

SPERMATAGONIAL STEM CELLS. The inventors of the present disclosure have advantageously identified three distinct populations of cells within the seminiferous tubules. The first population will be termed the “spermatogonial stem cells,” (SSC's) which are pluripotent cells that may ultimately differentiate into spermatozoa. The SSC's may be distinguished from other cell types within the seminiferous tubules by their expression patterns of various markers. SSC's express Stage Specific Embryonic Antigen-4 (“SSEA-4”), a glycosphingolipid found on the cell surface. SSC's do not express detectable levels of THY1 (a.k.a. CD90) surface protein. SSC's are dimly stained when stained with VASA antibodies. SSC's express little or no Vimentin, as described below. SSC's express high levels of various markers of pluripotency (TERT and LIN28B), putative SSC identity (ZBTB16, GFRA1, SALL4, MAGEA4, GPR125), and self-renewal (GFRA1, RET, ETV5), while expressing none or low levels of meiosis (DMC1, SYCP3) or spermatid differentiation (PRM2, ACR) markers.

MULTIPOTENT TESTICULAR STROMAL CELLS. The inventors of the present disclosure have further identified a second population of cells in the seminiferous tubule which will be referred to herein as multipotent testicular stromal (“MTS”) cells. The MTS cell population is a heterogeneous population of cells of mesenchymal origin. In one embodiment, MTS may be identified by their display of THY1 and the absence of SSEA-4. Testicular somatic cell cells display very low or no DAZL and VASA. Testicular somatic cells also exhibit brighter VASA staining than EGC cells and express higher levels of Vimentin.

The third population of cells present in the seminiferous tubules will be termed the “Differentiating Germ Cell” population (“DGC”). Cells of the GDC cell population express neither SSEA-4 nor THY1. The GDC population is also distinct from the SSC and MTS populations in that it contains haploid cells and express high levels of both meiotic (DMC1, SYCP3), and spermatid markers (PRM2, ACR). DGC cells also express very low levels of SSC markers ZBTB16, GFRA1, and GPR125.

CELL IDENTIFICATION USING MARKERS. The unique marker profiles of the three cell populations identified herein may be used as a basis to identify cells from each of the three populations. In one aspect, the invention comprises the identification of a cell from a seminiferous tubule tissue sample by its expression, or lack of expression, of one or more markers, wherein the cell is identified as an SSC cell, an MTS cell, or an EDG cell based on its expression or lack of expression of the one or more markers matching the expression pattern of such one or more markers as observed in the SSC, MTS, or DGC populations.

Marker presence or absence may be assessed by any means known in the art for the qualitative or quantitative measurement of gene activity or protein expression. For example, marker presence or absence may be assessed by measurement of gene expression activity, for example by quantitative PCR methodologies. Marker presence or absence may also be assessed by the use of labeled antibodies to proteins, for example, by the use of antibodies linked to fluorescently labeled proteins. It will be understood that reference to a fluorescently labeled antibodies herein further encompasses primary and secondary antibody labeling systems, e.g. wherein a primary antibody directed to a cellular antigen is adhered to the target moiety and then a second, fluorescently labeled antibody with affinity to the primary antibody is bound to the target antigen-bound primary antibody.

In one embodiment, SSC and MTS cells may be differentiated from each other by their relative expression of various markers, for example, as set forth in Tables I, II and III below. Table I lists 34 markers which are significantly differentially expressed between SSC and MTS cell populations and which such differential expression may be used as a basis to distinguish cells from the two populations from each other. Table II lists genes which are upregulated in SSC cells relative to MTS cells. Table III lists genes which are downregulated in SSC cells relative to MTS cells.

In one aspect, the marker profiles may be utilized to identify cells from each population in biopsied tissues, in cell cultures, or in isolated single cells or cell clusters. For example, in one embodiment, the presence or absence of SSEA-4 and THY1, which are displayed on the cell surface and are readily accessible to labeled antibodies, may be used for the facile identification of cells from each of the three populations, with SSC's being SSEA-4+ and THY1−, MTS cells being SSEA-4− and THY1+, and DGC's being SSEA-4− and THY1−. The invention further comprises the use of other marker profiles disclosed herein to differentiate cells from the three populations, for example by the presence or absence of specific markers or the relative abundance of the markers.

ISOLATION OF TESTICULAR CELL POPULATIONS. In another aspect, the invention comprises methods for the isolation of cells from each the three populations described above, for example from a heterogeneous group of cells isolated from a testicular tissue sample, i.e. the seminiferous tubules. The cell isolation procedures of the invention encompass any cell separation or sorting technology known in the art, for example fluorescence activated cell sorting (FACS) or similar flow-cytometry methodologies, magnetic-activated cell sorting, microraft sorting, affinity-based cell separation methods, and other means of isolating specific cell types from a mixed population of cells.

In one embodiment, the isolation process encompasses the use of (1) a heterogeneous sample of cells isolated from the seminiferous tubules; (2) a cell sorting system; and (3) a selected differentiating criteria comprising marker profiles unique to each population of cells. The unsorted cell sample is obtained from testicular tissue using biopsy methods known in the art. The testicular tissue may be subjected to any treatment known in the art for the liberation of single cells from the tissue, for example by enzymatic and/or mechanical processes. For example, an enzymatic digestion as described in Example 1 may be employed. Next, labels (e.g. antibodies labeled with fluorescent proteins) with specificity for markers corresponding to the selected differentiating criteria are applied or introduced to the isolated cells, such labels being compatible with the selected cell sorting system (e.g. fluorescent labels for FACS). As known in the art, labeling of either extracellular or intracellular marker proteins may be performed. Lastly, the cell sorting system is used to isolate cells from one or more of the populations present in the heterogeneous sample.

For example, in one embodiment, a FACS system is utilized as the cell sorting system with fluorescently-labeled antibodies to the extracellular SSEA-4 and THY1 markers applied to the heterogeneous cell mixture. Cells belonging to the SSC, MTS, and DGC populations are readily separated by their differential expression of these two markers, with SSC cells being SSEA-4+/THY1−, MTS being SSEA-4−/THY1+, and DGC cells being SSEA4−/THY1−. This method advantageously utilizes just two extracellular labels, simplifying sorting and minimizing potential disruptions of cell function. However, it will be understood that any other combination of the markers delineated herein may be used to differentiate cells from the three populations, for example, including intracellular markers and sorting of cells based on relative expression of markers, enabled by intracellular labeling and quantitative FACS methodologies, as known in the art.

In one embodiment, the invention comprises a kit comprising two or more fluorescently labeled antibodies wherein the antibodies preferentially bind two or more gene products (i.e. proteins) derived from genes which are differentially expressed in SSC and MTS cells, and wherein the fluorescent labels are distinguishable from each other. In one embodiment, the kit comprises an antibody to SSEA-4 and an antibody to THY1.

CULTURING AND EXPANSION OF SSC's. In another aspect, the invention comprises methods for the expansion of SSC's isolated from testicular tissue samples. As set forth in Example 1, the inventors of the present disclosure have elucidated the niche required for SSC culture in vitro. Specifically, the co-culture of cells from the SSC and MTS cell populations is required to promote the efficient expansion of SSC's. SSC's adhere to MTS cells, which presumably provide them with essential growth factors which promote their growth and renewal.

In one embodiment, the invention comprises the co-culture of SSC and MTS on a cell culture substrate. Exemplary substrates include bare culture dish surfaces, or culture vessels coated with cell culture substrates or feeder cell layers known in the art, for example Matrigel, gelatin, irradiated mouse embryonic fibroblasts, human placental fibroblasts, and human fetal testicular stroma. Additional culture substrates include xeno-free substrates such as SYNTHEMAX™ (Corning), CELLSTART™ (Life Technologies), recombinant parylene, recombinant poly-lysine D, and other xeno-free substrates known in the art. Alternatively, solution culture may be utilized. The cell culture methods of the invention are enabled by the use of a suitable culture medium, including any culture medium known in the art for the culture of stem cells in general or SSC's specifically. An exemplary medium includes KnockOut™ DMEM or DMEM/F12, with 20% KOSR, 1% non-essential amino acids, 1×GLUTAMAX™ (Invitrogen) supplement and 4-10 ng/mL FGF2, as described in Example 1.

Co-culture of SSC's and MTS cells gives rise to SSC colonies and may be achieved by mixing isolated cells from each population, for example in a ratio of 1:1, and then plating the mixed cells.

In one embodiment, the SSC's are cultured on a layer of MTS cells. MTS cells, being highly adherent, are first cultured on a substrate. When a layer of MTS cells has been established on the substrate, the SSC cells may be added to the culture vessel, wherein some of them will adhere to the underlying MTS cells and will start to form colonies. SSC's can then be expanded by harvesting, passaging the harvested cells onto fresh layers of MTS cells, and allowing new SSC colonies to form and grow. The SSC colonies formed on MTS cells may be repeatedly passaged, for example being passaged every 10-20 days, for example, every 14 days, to fresh MTS cell-coated culture substrates, in order to propagate and expand SSC numbers.

The inventors of the present disclosure have advantageously discovered that in vitro culture conditions tend to favor the growth of MTS cells over SSC cells. This differential growth in culture can lead to the overgrowth of MTS and disappearance of co-cultured SSC colonies. Accordingly, the invention further encompasses methods of improving the relative performance of SSC's co-cultured with testicular somatic cells. In one embodiment, the MTS cells are treated with chemical or radiation treatments to inhibit their division/expansion, for example being treated with chemical or radiation treatments which render the cells mitotically compromised or mitotically inactive. For example, MTS cells that have reached confluence or nearly reached confluence in a culture vessel may be treated with chemical or radiation treatments to render them mitotically inactive or compromised. For example, as described in Example 1, the MTS cells may be exposed to a radiation treatment to inhibit their rate of division and growth, prior to their co-culture with SSC's. For example, irradiation by gamma radiation may be performed. For example, irradiation at doses of 2,500-3,5000 rads may be performed. This treatment inhibits the growth of the MTS cells and advantageously allows SSC's to grow and expand at high rates without being outcompeted by MTS cells.

The culture of human SSC's on MTS cells may comprise an autologous system, wherein the MTS cells are derived from the same person as the SSC's plated thereon. In an alternative embodiment, an allogenic culturing system is utilized wherein SSC's are cultured on MTS cells derived from another person.

METHODS OF CREATING SPERMATOZOON AND RESTORATION OF FERTILITY. In one aspect, the invention encompasses the isolation and expansion of SSC's derived from a person for the subsequent formation of spermatozoon. In one embodiment, isolated SSC's are expanded, as described above. Such expanded SSC's may be cryopreserved, utilizing cryopreservation techniques known in the art, for example as described in Jahnukainen et al., “Effect of cold storage and cryopreservation of immature non-human primate testicular tissue on spermatogonial stem cell potential in xenografts,” Human Reproduction. 2007; 22:1060-1067; and Lee et al., “Cryopreservation of mouse spermatogonial stem cells in dimethylsulfoxide and polyethylene glycol,” Biol Reprod. 2013 November 7; 89(5):109. The SSC's may subsequently be utilized for the in vitro formation of spermatozoon, utilizing methods known in the art for in vitro spermatogenesis, for example as described in Sato et al., (2011) in vitro production of functional sperm in cultured neonatal mouse testes. Nature 471: 504-507; and Abu-Elhija et al., (2011) Differentiation of murine male germ cells to spermatozoa in a soft agar culture system. Asian J Androl 1-9. In one embodiment, the invention comprises a composition of matter comprising spermatozoon produced from SSC's which have been previously cultured in vitro on MTS cells.

Spermatozoon produced from in vitro expanded SSC's may be utilized in in vitro fertilization techniques, intracytoplasmic sperm injection, in vivo fertilization, and other methods known in the art for fertilization using spermatozoon. In one aspect, the invention comprises a method of fertilizing an egg cell by means of spermatozoon derived from SSC's cultured and expanded in vitro on MTS cells.

In another embodiment, SSC's are isolated from a male subject, are expanded in vitro, and, subsequently (e.g. after cryopreservation and storage), are transplanted back into the testes of the donor. Methods of transplantation amenable with the cells of the invention are known in the art, for example as described in Radford J., “Restoration of fertility after treatment for cancer,” Horm Res. 2003; 59(Suppl 1):21-23; Jahnukainen et al. “Testicular recovery after irradiation differs in prepubertal and pubertal non-human primates, and can be enhanced by autologous germ cell transplantation,” Hum Reprod. 2011; 26:1945-1954; and Schlatt S, Foppiani L, Rolf C, Weinbauer G F, Nieschlag E. Germ cell transplantation into X-irradiated monkey testes. Hum Reprod. 2002; 17:55-62.

MALE FERTILITY GENES. Table IV lists genes commonly expressed in mouse gonocytes, human prepubertal SSCs, and human adult SSCs. The highly conserved nature of these genes indicates that they are important for male fertility, and the genes will hereafter be referred to as “highly conserved SSC genes.” Accordingly, in one aspect, the invention comprises methods of diagnosing infertility or fertility problems by the detection of aberrant expression of one or more highly conserved SSC genes listed in Table IV. In one embodiment, aberrant expression comprises expression which deviates from normal wild type expression level, e.g. deviates lower or higher than wild type expression by, for example, 10%, 25%, 50%, 100% or more. In another embodiment, aberrant expression comprises an expression level relative to that in THY1 positive cells which deviates substantially lower or higher (e.g. 10%, 25%, 50%, 100% or more lower or higher) from the relative expression levels indicated in Table IV. Detection of aberrant expression is accomplished by obtaining a testicular tissue sample from a male subject, and then measuring the expression of one or more genes from Table IV, such detection accomplished utilizing any method known in the art, for example by measuring gene expression in SSC's in situ in the testicular tissue, or by isolating the SSC's and assessing gene expression in the isolated SSC's. In one embodiment, one or more highly conserved SSC genes is sequenced, either at the genomic or transcript level, in order to assess mutation status, with mutant forms being indicative of infertility. In one embodiment, the invention comprises a microarray or qtPCR probe set comprising probes complementary for two or more highly conserved SSC genes. In another embodiment, the invention comprises fertility treatment by restoring normal patterns of expression of one or more genes in Table IV in a male subject where aberrant expression of one or more highly conserved SSC genes is found. In another embodiment, the invention comprises fertility treatment by complementing mutant forms of genes from Table IV present in a patient by introducing wild-type forms of the mutant gene(s) or gene product(s). The therapeutic methods of the invention are accomplished by gene therapy or SSC transplantation techniques, as known in the art.

Reference to genes and/or markers herein utilize the common and known abbreviations/codes for human gene sequences, as known in the art. For convenience, the description herein is directed to “persons” and “male humans,” however, it will be understood that the scope of the invention extends to males from other species, for example, the practice of the isolation and expansion methods of the invention utilizing orthologous and/or homologous genes from non-human species such as equine, canine, feline, murine, and non-human primates. The scope of the invention applies to research, medical, and veterinary uses.

Exemplary embodiments of the invention are disclosed and illustrated in the following Example 1.

### Example 1

Two approaches may potentially help pre-pubertal boys become fathers after cancer treatment. Testicular tissue taken prior to chemotherapy could be differentiated into mature sperm. This approach combined with in vitro fertilization (IVF) and intracytoplasmic sperm injection (ICSI) has been successful in a neonatal mouse model. Alternatively, autologous spermatogonial stem cell (SSC) transplant has been shown to restore spermatogenesis leading to healthy offspring in many non-primate models for more than 15 years and, most recently, in primates. However, neither approach has been attempted in humans.

Despite advances in fertility treatment that have led to the routine use of IVF/ICSI for men with ejaculated or surgically obtained sperm concentrations approaching zero, these techniques are not possible for pre-pubertal boys as they do not produce sperm. However, their testicles do contain SSCs with the potential to expand and differentiate into mature sperm. Thus, techniques to expand SSCs for autologous transplantation or to differentiate SSCs into mature sperm are of tremendous value. Challenges include the identification, isolation, and expansion of highly purified SSCs due primarily to a gap of knowledge in identifying human SSCs based on extracellular marker expression and understanding the cellular environment (“niche”) necessary for SSC growth and differentiation.

Expansion of purified mouse SSCs using unique membrane protein markers such as Thy-1, GFRA1, GPR125, and CD49f have been reported. Allogeneic transplantation of in vitro expanded mouse SSCs into germ cell depleted testes resulted in restoration of fertility. Recent studies propose that human SSCs may be identified based on expression of THY1, CD49f, EPCAM, and SSEA-4. Of these markers, only SSEA-4 is highly expressed in embryonic stem cells and in both human fetal and prepubertal SSCs suggesting that it may be a good marker of adult human SSCs. Although transplantation of human testicular cells into germ cell depleted mouse testes resulted in either limited colonization of human cells or cells expressing germ cell markers, spermatogenesis was not detected, presumably due to interspecies differences. Mouse and human SSCs have been reported to have the capability of converting into testis-derived pluripotent stem cells during in vitro culture. However, in contrast to studies in mice, recent studies suggest that the human testis-derived pluripotent stem cells derived from in vitro culture of putative human SSCs are actually cells of mesenchymal rather than germ cell origin. Filling this gap in the understanding of human SSC biology is important.

Due to the limited availability of human tissues, the lack of in vitro or in vivo xenograft models capable of supporting human spermatogenesis, and the significant ethical and logistical challenges associated with human germ cell research, current data on the identification, isolation, and expansion of human SSCs are mixed and highly controversial. To shed light on this controversy and lay the groundwork for a new therapy for young male patients facing sterilizing treatments, a detailed characterization of SSCs and the required somatic niche capable of supporting SSC expansion is needed.

Subjects:

Thirteen adult subjects with normal spermatogenesis were enrolled in the IRB approved, University of California San Francisco LIFE and Human SSC studies.

Testicular Cell Isolation:

Tissues obtained by testicular biopsy were subjected to a two-step enzymatic digestion with collagenase IV (1 mg/ml) in DMEM/F12+Glutamax (Invitrogen, Carlsbad, Calif.) for 20 min at 37° C., followed by trypsin EDTA (0.25%) (UCSF Cell Culture Facility (CCF), San Francisco, Calif.) and DNase I (50 μg/ml) (Sigma-Aldrich, St. Louis, Mo.) for 20 min, and filtered through a 70 μm cell strainer.

Fluorescence Activated Cell Sorting (FACS):

Biopsied tissues and digested and cells were incubated with the following antibodies: anti-SSEA-4 FITC, anti-THY1 APC, anti-CD105 FITC, and anti-CD73 PE in 1% bovine serum albumin (BSA) for 30 min at 37° C. (all from BD Pharmingen, San Jose, Calif., USA).

Prior to sorting, only live singlet cells were gated for analyses. Cell sorting was performed on a BD FACS Aria Flow Cytometer and analyzed using FlowJo v9.6. DNA content was assessed by staining 70% ethanol-fixed cells in 0.1% Triton X-100, 20 μg/ml propidium iodide, 200 μg/ml RNase A (Sigma-Aldrich) for 15 min at 37° C. Cells were analyzed on a BD LSR II flow cytometer. For mesenchymal studies, testicular cells were stained with THY1 APC, CD73 PE, 105 FITC, CD2, 3, 16, 45-Biotin-Pacific Blue antibodies and subjected to flow cytometric analyses. Dead cells and cells expressing CD2, 3, 16, and 45 were excluded. Only singlet THY1+ cells were gated for further analyses.

Confocal Microscopy:

Images were captured using a Leica SP5 AOBS confocal microscope, Leica DMI 4000B fluorescent microscope (Leica Microsystems Inc., Bannockburn, Ill.), and analyzed using ImageJ v1.6.

Testicular tissues were fixed in 4% paraformaldehyde (PFA), embedded in optimal cutting temperature compound (O.T.C) (Sakura Finetek, Torrance, Calif.), and cryosectioned at 5 μm. Sections and cultured cells were permeabilized with 0.5% Triton-X-100 PBS, blocked in 5% BSA-PBS, and incubated overnight at 4° C. with the following antibodies: anti-VASA, anti-THY1 (R&D Systems, Minneapolis, Minn.), anti-WT1 (Santa Cruz Biotechnology, Santa Cruz, Calif.), anti-CD-73, anti-CD-105, and anti-SSEA-4 at 1:50 to 1:500 dilutions. Secondary antibodies: donkey anti-goat Alexa 555, donkey anti-sheep Alexa 555, donkey anti-rabbit Alexa 647, donkey anti-rabbit Alexa 555, and donkey anti-mouse Alexa 488 (BD Pharmingen) were applied the following day (1:500 dilution) at room temperature for 1 hr.

Molecular Analyses:

Subpopulations of testicular cells were sorted directly into RNA buffer. Total RNA was isolated using the RNeasy Micro Kit (QIAGEN, Valencia, Calif.) and cDNA synthesized using qScript cDNA Super Mix (Quanta Biosciences, Gaithersburg, Md.). qPCR amplification was carried out using FastStart Universal SYBR Green Master Mix with ROX (Roche, Mannheim, Germany) using 7500 PCR system (Applied Biosystems). Sorted THY1+, SSEA-4+, and THY1−/SSEA-4− cells (100 cells/reaction) from each patient (n=3) were run in triplicates and normalized to glyceraldehyde-3-phosphate dehydrogenase (GAPDH) expression. Levels of gene expression were analyzed using the 2^{-(ΔC(t))} and 2^{-(ΔΔC(T))}. ANOVA and Student t-tests were used for statistical analyses.

The CEL files of human and mouse microarray data were obtained from Gene Expression Omnibus (accession no. GSE18914). The CEL files included expression data of 6 human samples (3 samples each of human prepubertal spermatogonia and testis somatic cells) profiled using Affymetrix Human Genome U133 Plus 2.0 Array and 6 mouse samples (3 samples each of mouse gonocytes and testis somatic cells) profiled using Affymetrix Mouse Genome 430 2.0 Array. Four hundred cells were selected for each group of germ cells or testis somatic cells from three independent testis cell preparations from both human (ages 2, 8 and 10 years) and mouse (age 3 days).

The CEL files were loaded in affy package in R and Robust Multi-array Average (RMA) algorithm was used to generate an expression matrix from the CEL files. The raw intensities values were background corrected, log₂ transformed, and quartile normalized for further analysis. Linear Models for Microarray data (LIMMA) package was applied on normalized expression value to obtain differential expressed genes. Genes with at least log₂±2 fold-changes in expression and adjusted p-value <0.05 were considered differentially expressed. A total of 125 genes were differentially expressed between mouse gonocytes and somatic cells and 1049 genes were differentially expressed between human spermatogonia and somatic cells.

RNA-seq data was generated from FACS sorted human testicular THY1+ cells and SSEA-4+ cells obtained from three healthy individuals. Briefly, THY1+ and SSEA-4+ cells were sorted directly into RNA buffer. Purified RNA was analyzed for quality using chip-based capillary electrophoresis (Bioanalyzer, Agilent, Inc, Santa Clara, Calif.) and quantity and purity was determined with a NanoDrop spectrometer. RNA-seq libraries were prepared with ovation RNA-seq system v2 kit (NuGEN, San Carlos, Calif.). In this method, the mRNA is reverse transcribed to synthesize the first-strand cDNA using a combination of random hexamers and a poly-T chimeric primer. The RNA template is then partially degraded by heating and the second strand cDNA is synthesized using DNA polymerase. The double-stranded DNA is then amplified using single primer isothermal amplification (SPIA). SPIA is a linear cDNA amplification process in which RNase H degrades RNA in DNA/RNA heteroduplex at the 5′-end of the double-stranded DNA, after which the SPIA primer binds to the cDNA and the polymerase starts replication at the 3′-end of the primer by displacement of the existing forward strand. Random hexamers are then used to amplify the second-strand cDNA linearly. Finally, libraries from the SPIA amplified cDNA were made using the Ultralow DR library kit (NuGEN). The RNA-seq libraries were analyzed by Bioanalyzer and quantified by qPCR (KAPA). High-throughput sequencing was done using a HiSeq 2500 (Illumina).

The read counts were calculated from RNA-seq data using HTseq count package and then DESEq package was applied on read counts to obtain differentially expressed genes. The read counts were normalized by scaling (by library size), dispersion was estimated and negative binomial distribution was fitted to the normalized data. Genes with at least log₂±2 fold-changes in expression p-value <0.05 were considered differentially expressed. Differentially expressed genes from the above 3 datasets (RNA-seq, human microarray, and mouse microarray data) were analyzed for overlap. Expression data is presented in Tables I, II, III, and IV.

IPA was performed on common up-regulated genes to identify the molecular pathways and functional groupings. Gene networks were generated by IPA with score >25 were selected for presentation.

SSC Culture:

Subpopulations of testicular cells were FACS sorted and individually cultured in supplemented StemPro-34 (Invitrogen) with the following modifications: 1% penicillin-streptomycin, 1% ITS solution (Mediatech Inc, Manassas, Va.), recombinant human EGF (20 ng/mL) (R&D Systems), recombinant human GDNF (10 ng/mL) (R&D Systems), recombinant human LIF (10 ng/mL) (Chemicon International Inc, Temecula, Calif.) and 1% KOSR (knockout serum replacement). Media was changed every 72 hours. These culture conditions are exemplary, and certain variations are specifically contemplated. For example, in alternative embodiments, substantially similar products from different manufacturers may be used. In addition, the concentrations of the culture media components may be adjusted slightly as a matter of preference. For example, the concentration of penicillin-streptomycin may range from 0.5-2%. The concentrations of GDF and/or LIF may range, for example, from 10-20 ng/mL. Media may be changed every 48-72 hours. Cells were cultured at 37° C. in a humidified incubator with 5% CO₂ and 20% O₂ without stromal cells on either Matrigel or 0.2% gelatin coated dishes at a density of 50,000 cells/cm² and on either mouse embryonic fibroblasts (MEFs), human fetal placental fibroblasts, human fetal testicular fibroblasts, or human adult testicular THY1+ cells at a density of 25,000 cells/cm². Human adult testicular THY1+ cells that support SSC growth were cultured in KnockOut™ DMEM or DMEM/F12, with 20% KOSR, 1% non-essential amino acids, 1× GlutaMAX™ supplement (all from Invitrogen) and 4-10 ng/mL FGF2. These cells were passaged 3-5 times and gamma irradiated using a cesium-source irradiator for 3000 rad. Human fetal placental and testicular fibroblasts at 22 weeks of gestation were generated from discarded fetal tissues donated for research.

Characterization of Testicular Multipotent Stromal Cells:

Testicular THY1+, CD73+, CD105+ cells were isolated by FACS and expanded in DMEM-low glucose with 2 mM glutamine, 1% penicillin/streptomycin, and 10% FBS, and differentiated using Hyclone ADVANCESTEM™ Chondrogenic Differentiation Medium (Thermo Fisher Scientific, Waltham, Mass.), Mesenchymal Stem Cell Adipogenesis Kit (Millipore, Billerica, Mass.) and STEMPRO™ Osteogenesis Differentiation Kit (Invitrogen) according to manufacturer's instructions. Chondrogenic differentiation was confirmed with Alcian Blue (pH 2.5) staining. Lipid deposits indicative of adipogenic differentiation were detected with Oil red O. Calcium deposits detected with Alizarin Red stain indicated osteogenic differentiation.

### Characterization of SSC's and MTS Cells

Testicular THY1+ and SSEA-4+ Cells Represent Distinct Cell Populations:

In the prior art, both THY1 and SSEA-4 were reported as putative markers of adult human SSCs. However, while recent studies demonstrate that both THY1 and SSEA-4 are expressed in fetal gonocytes, only SSEA-4 continued to be expressed in prepubertal SSCs. Thus, confocal microscopy was performed to detect the presence and precise location of cells expressing THY1, SSEA-4, and VASA. Two distinct populations of germ cells were observed by relative expression of VASA. VASA dim cells, located at the basement membrane, correlate anatomically with the known location of SSCs. In contrast, VASA bright cells, located predominantly away from the basement membrane toward the lumen, are consistent with differentiating spermatogonia, spermatocytes, and spermatids. SSEA-4 expression was primarily detected in VASA dim cells on the basement membrane suggesting that it is a specific marker for SSCs Additionally, evidence of meiosis, assessed by SYCP3 expression, was exclusively detected in VASA bright cells suggesting that SSEA-4/VASA dim population contain adult human SSCs. In contrast, THY1 expression was detected primarily on fibroblasts and myoid cells of the lamina propria with limited expression on peritubular cells. Additionally, the cell population located between the VASA dim and bright cells exclusively expressed both THY1 and WT1 consistent with Sertoli cells. All THY1+ cells expressed high levels of Vimentin and did not express VASA suggesting they are likely of somatic and mesenchymal origin.

Seminiferous tubules were digested and testicular cells were dual stained with THY1 and SSEA-4 antibodies for further characterization of the THY1+ and SSEA-4+ populations by FACS. Three distinct populations of testicular cells based on THY1 and SSEA-4 expression (THY1+, SSEA-4+, and THY1−/SSEA-4− populations) were observed. There were no cells that co-expressed both THY1 and SSEA-4 simultaneously. When THY1+, SSEA-4+, and THY1−/SSEA-4− populations were analyzed separately by backgating to examine their unique cellular characteristics, each demonstrated distinct forward and side scatter values providing further confirmation that these three populations possessed different cellular physical properties. Immunofluorescent analyses of the sorted THY1+, SSEA-4+, and THY1−/SSEA-4− populations demonstrated the lack of SSEA-4, THY1, and either THY1/SSEA-4 expression in these populations, respectively.

Sorted THY1+, SSEA-4+, and THY1−/SSEA-4− cells were subjected to DNA content and mRNA analyses. Significant amount of haploid (N) and tetraploid (4N) cells, 11% and 48%, respectively, were identified exclusively in the THY1−/SSEA-4− population suggesting that only the THY1−/SSEA-4− population contained differentiating germ cells. In addition to the lack of haploid cells, both THY1+ and SSEA-4+ populations contained a very small population of 4N cells, 5% and 9%, respectively, suggesting that they are mainly quiescent in normal homeostatic state. Although expression of DAZL and VASA were detected in the SSEA-4+ and THY1−/SSEA4− cell populations, they were barely detectable in the THY1+ cells. Instead, THY1+ cells were found to express high levels of VIM, >98 and 27 fold over SSEA-4+ and THY1−/SSEA-4− cells, respectively, suggesting a mesenchymal origin. While both SSEA-4+ and THY1−/SSEA-4− populations expressed germ cell markers (DAZL and VASA), only the THY1−/SSEA-4− population contained haploid cells and expressed high levels of both meiotic (DMC1, SYCP3), and spermatid markers (PRM2, ACR) demonstrating that SSEA-4+ population contains primitive spermatogonia that have not yet entered meiosis. As expected, very low levels of known mouse SSC markers, ZBTB16, GFRA1, and GPR125 were detected in the THY1−/SSEA-4− population. Although both THY1+ and SSEA-4+ populations expressed ZBTB16, GFRA1, and GPR125, the expression was significantly higher in the SSEA-4+ population, assessed by qPCR, and confirmed with FACS.

### Characterization of the Niche Required for SSC Expansion

Establishment of SSC Colonies from Unsorted Adult Testicular Cells:

During the first three days of culture, ˜10-15% of cells began to adhere to the culture dish, exhibited fibroblast morphology, grew, and expanded in a monolayer fashion, independent of whether the dish was uncoated or coated with Matrigel or gelatin. Subsequently, ˜5% of the remaining non-adherent cells began binding to these adherent cells and form colonies. The remaining non-adherent cells slowly died between 14-21 days of culture. Three early SSC colonies bound exclusively to the fibroblast like cells and slowly expanded in size. These colonies continued to express SSEA-4 and VASA. However, the proliferation rate of the fibroblast like cells far exceeded SSC's rate. Once >75% confluent, these fibroblast like cells outgrew and inhibited the growth of SSC colonies. Early passages did not rescue SSC colonies as the fibroblast like cells continued to outgrew SSCs.

Sorted Testicular THY1+ Cells Exhibited Fibroblast Like Morphology:

Sorted testicular THY1+ cells quickly bound to culture plates and grew in a monolayer fashion regardless of whether they were uncoated, coated with Matrigel or gelatin, or supported by different types of irradiated stromal cells. Sorted primary THY1+ cells cultured in the presence of irradiated THY1+ cells indiscriminately bound to plastic or the irradiated THY1+ cells within 48 hours of culture, established fibroblast like morphology, and grew in a monolayer fashion without ever forming SSC colonies. They continued to grow in this fashion despite many passages.

Sorted Testicular SSEA-4+ Cells Gave Rise to SSC Colonies in the Presence of Irradiated Testicular Thy-1+ Cells:

Sorted testicular SSEA-4+ cells were plated on culture dish with irradiated testicular THY1+ cells plated 48 hours prior. SSEA-4+ cells tend to bind to either adherent THY1+ cells or to each other. However, only SSEA-4+ cells that bound to the adherent THY1+ cells established SSC colonies and grew in size. The remaining nonadherent SSEA-4+ cells ceased to grow and died in cultures between 14-21 days of culture. SSEA-4+ cells failed to bind to plates coated with Matrigel, gelatin, irradiated mouse embryonic fibroblasts, human placental fibroblasts, and human fetal testicular stroma.

Passages of Human SSC Colonies Established from Sorted Testicular SSEA-4+ Cells:

Established SSC colonies were passaged every 2 weeks onto new irradiated testicular THY1+ cells. Single SSCs quickly bound to adherent THY1+ cells and formed new colonies. The adherent THY1+ cells exhibited some migration in culture. However, the SSC colonies also migrated along with their adherent cells, demonstrating the importance of the interactions between SSCs and the niche provided by THY1+ cells.

Testicular THY1+ Cells are Critical for Successful SSC Expansion:

Unsorted, sorted THY1+, and sorted SSEA-4+ cells were subjected to in vitro expansion and monitored with time-lapse photography. Unsorted testicular cells cultured on either uncoated or coated plates revealed two populations. The first adhered to the plates and exhibited fibroblast like morphology within 48 hours. The second population of small round cells bound to these fibroblast-like adherent cells shortly after 48 hours, divided, and formed colonies after 2 weeks of culture. However, colonies began to disappear after 3 weeks of culture as the adherent cells became confluent. Although ˜98% of these in vitro expanded unsorted testicular cells expressed THY1, evaluated by FACS, after 3 weeks of culture, neither SSEA-4 nor VASA expression was detected by FACS, microscopy, or qPCR. Cell passage after 2 weeks of culture did not rescue expansion of SSC colonies as the adherent cells quickly grew to confluence suggesting a preferential selection of THY1+ cells in this culture system.

When plated on culture dishes uncoated or coated with either Matrigel or gelatin, THY1+ cells adhered to all plates within 24 hours, exhibited fibroblast morphology shortly after, and continue to expand without signs of quiescence (>20 passages). Although DAZL and VASA/VASA were never detected by qPCR or confocal microscopy, this population continued to express high levels of THY1 and Vimentin, assessed by immunofluorescent analyses. In contrast, SSEA-4+ and THY1−/SSEA-4− cells did not adhere or form colonies when cultured on uncoated or coated plates, failed to expand, and died within 2 weeks of culture. Furthermore, immunofluorescent analyses did not detect any evidence of THY1 and Vimentin expression in these two populations.

To overcome the rapid expansion of THY1+ cells in this system, sorted THY1+ cells were expanded and subjected to γ-irradiation to render them mitotically inactive. Sorted SSEA-4+ cells were then co-cultured on the irradiated adherent THY1+ cells. SSEA-4+ cells bound to these adherent cells within 24 hours, formed SSC colonies (˜50 cells/colony) within 2 weeks, and continued to expand. The percentage of SSC colonies formed to SSEA-4+ cells plated ranged between 0.02-0.1% with an 8-12 fold increase in colony number and cell number (50-100 cells/colony) after each subsequent passage. These expanded colonies continued to express SSEA-4 and VASA with serial passaging. In contrast, THY1−/SSEA-4− cells failed to establish colonies when plated on irradiated THY1+ cells. Additionally, THY1+, SSEA-4+, and THY1−/SSEA-4− cells failed to establish colonies when cultured in the presence of MEFs, human placental, or fetal testicular stroma. Thus, adult testicular THY1+ cells were found to uniquely provide the essential niche required for SSC expansion. Using this novel system, SSC colonies were successfully identified, isolated, passaged, and expanded in vitro.

Testicular THY1+ Cells Demonstrated Mesenchymal Properties:

In addition to their lack of germ cell properties, sorted THY1+ cells immediately adhered to plastic and exhibited fibroblastic morphology suggesting a mesenchymal origin. To investigate whether the THY1+ population exhibited mesenchymal characteristics, THY1+ cells were analyzed for co-expression of CD73 and CD105. 92% of THY1+ cells co-expressed both CD73 and CD105. When THY1+ cells were expanded in vitro, they continued to co-express both CD73 and CD105.

Upon differentiation, sorted THY1+/CD73+/CD105+ cells gave rise to adipocytes, chondrocytes, and osteocytes further confirming the presence of mesenchymal properties within this population. Similarly, while VIM was highly expressed in the THY1+/CD73+/CD105+ population, neither DAZL nor VASA were detected in this population.

### Comprehensive Molecular Characterization of the Testicular THY1+ and SSEA-4+ Cells by Next Generation Sequencing

Testicular SSEA-4+ Cells Expressed Genes Previously Identified as Enriched in Human and Mouse SSCs:

FACS sorted THY1+ and SSEA-4+ cells were collected from three patients and subjected to mRNA sequencing. On average, there were 13,568,327 and 8,822,058 total reads from the THY1+ and SSEA-4+ populations, respectively. In principal component analysis, the THY1+ and SSEA-4+ populations are significantly distinct from each other. While the SSEA-4+ population cluster tightly, the Thy-1+ population show more variability reflecting this population's innate heterogeneity. When log₂±2 fold-change with p-value <0.05 was used as the cutoff to define significant differential gene expression, there were 1359 known up-regulated and 1911 down-regulated genes in the SSEA-4+ population in comparison to the THY1+ population. Specifically, 29 genes were up- and 232 were down regulated >100 fold. Genes previously reported to be enriched in human and mouse SSCs were examined. Table I demonstrates the enriched expression of known SSC genes in testicular SSEA-4+ cells. When further evaluated by FACS, EPCAM, GPR125, and ITGA6/CD49f were found to express in both THY1+ and SSEA-4+ populations (Table I). However, qPCR confirmed the higher expression of GPR125 in the SSEA-4+ population. Although KIT was differentially expressed in the SSEA-4+ population, c-KIT was not detected by confocal microscopy or FACS. Known intracellular markers of SSCs were highly enriched in the SSEA-4+ cells. Although ZBTB16 was not found to be enriched with mRNA-seq, qPCR data demonstrated that it was significantly enriched (1.9 fold) in the SSEA-4+ population. Additionally, known genes (RET, GFRA1, and ETV5) in the glial cell line-derived neurotrophic factor (GDNF) mediated SSC self-renewal pathway in rodent were also highly enriched in SSEA-4+ cells (Table I). Furthermore, known pluripotency markers such as TERT and LIN28B were highly expressed in the SSEA-4+ cells further suggesting that this population contains human SSCs. As expected and confirmed with qPCR, NANOG, SOX2, and POU5F1 were not expressed in any significant amount in either populations. In contrast, known somatic genes were highly expressed in the THY1+ population (Table I). The diverse families of somatic genes expressed in the THY1+ cells confirmed that this is a heterogeneous population. Overall, the testicular SSEA-4+ and THY1+ mRNA transcriptome profiles confirmed that the SSEA-4+ population contains primitive spermatogonia in contrast to the profile of the THY1+ population which favors a somatic origin.

TABLE I
Expression of genes in human testicular SSEA-4+ and THY1+ cells
previously identified as enriched in either human prepubertal or mouse
SSCs or somatic cells.

| | SSEA-4 | THY1+ | Fold | |
|---|---|---|---|---|
| Gene Symbol | Cells* | Cells* | difference** | P-value |
| Surface markers | | | | |
| KIT*** | 1613.35 | 46.74 | 35 | <0.001 |
| EPCAM*** | 504.85 | 120.08 | 4 | <0.001 |
| GPR125*** | 1176.55 | 426.3 | 3 | <0.001 |
| ITGA6 | 4105.03 | 2285.02 | 2 | 0.04 |
| Intracellular markers | | | | |
| SALL4 | 471.16 | 12.01 | 39 | <0.001 |
| MAGEA4 | 2383.7 | 102.56 | 23 | <0.001 |
| DAZL*** | 3539.63 | 291.16 | 12 | <0.001 |
| VASA*** | 4680.27 | 495.24 | 9 | <0.001 |
| NANOS2 | 338.83 | 37.16 | 9 | <0.001 |
| DNMT3B | 272.88 | 50.93 | 5 | <0.001 |
| ZBTB16*** | 78.96 | 86.87 | 1 | 1.00 |
| Self-Renewal Markers | | | | |
| RET | 438.9 | 6.96 | 63 | <0.001 |
| PIWIL2 | 2290.29 | 86.81 | 26 | <0.001 |
| GFRA1*** | 4814.8 | 489.06 | 10 | <0.001 |
| ETV5 | 1705.3 | 799.24 | 2 | <0.001 |
| Pluripotency markers | | | | |
| TERT | 69.05 | 0.37 | 187 | <0.001 |
| LIN28B | 806.19 | 80.56 | 10 | <0.001 |
| NANOG*** | 6.84 | 1.84 | 4 | 0.07 |
| SOX2*** | 1.11 | 0.37 | 3 | 1.00 |
| POU5F1*** | 12.09 | 6.16 | 2 | 0.83 |
| | SSEA-4 | THY1+ | Fold | P- |
| Gene Symbol | Cells* | Cells* | difference** | value*** |
| Somatic markers | | | | |
| ICAM | 65.64 | 14114.38 | 0.004 | <0.001 |
| ACTA2 | 95.71 | 22090.97 | 0.004 | <0.001 |
| ENG*** | 20.67 | 14987.49 | 0.004 | <0.001 |
| SNAI2 | 14.6 | 1966.71 | 0.007 | <0.001 |
| VIM*** | 290.86 | 37021.99 | 0.008 | <0.001 |
| NT5E*** | 12.67 | 971.34 | 0.01 | <0.001 |
| INSL3 | 15.70 | 1127.92 | 0.01 | 0.03 |
| CD34 | 124.89 | 5196.39 | 0.02 | <0.001 |
| THY1*** | 15.25 | 811.82 | 0.02 | <0.001 |
| GATA4 | 31.65 | 1001.3 | 0.03 | <0.001 |
| WT1*** | 11.42 | 408.39 | 0.03 | <0.001 |
| STAR | 67.04 | 2650.66 | 0.03 | 0.01 |
| CD44 | 81.65 | 2188.41 | 0.04 | <0.001 |
| AR | 124.09 | 2468.53 | 0.05 | <0.001 |

*Average normalized reads for expression by mRNA-seq of the three biological samples, normalized as described in Example 1:
**Fold difference between SSEA-4+ cells and Thy-1+ cells:
***Validated with qPCR, FACS, or confocal microscopy

Testicular SSEA-4+ Cells Expressed Genes Previously Identified as Enriched in Human Prepubertal SSCs and Mouse Gonocytes:

Since the results above support the hypothesis that testicular SSEA-4+ population contains adult human SSCs, it is important to compare this population to known pure populations of human or mouse SSCs. Hence, the transcriptome of the testicular SSEA-4+ cells reported here was compared to previously published human prepubertal SSC transcriptome as current information on adult human SSCs are limited and controversial. Thus far, only one study comparing human prepubertal SSC transcriptome to those of mouse gonocytes has been published. The RNA-seq transcriptome from this study was analyzed and compared with the microarray transcriptomes obtained from a published study, which performed microarrays in human prepubertal SSCs and testicular somatic cells (ages 2, 8, and 10) and compared them with mouse gonocytes and testicular somatic cells (age 3 days). The published study closely resembled this current study comparing adult human SSCs (SSEA-4+ population) to testicular somatic cells (THY1+ population). Since two different platforms (mRNA-seq vs. microarray) were used, PCA and hierarchical clustering were not feasible due to significant differences in sensitivity between the platforms. When log₂±2 fold-change with p-value <0.05 was used to define significant differential gene expression, there were 798 genes that were up-regulated and 251 that were down-regulated in the human prepubertal SSCs in comparison to their respected somatic population. In contrast, 112 genes were up- and 13 down regulated in the mouse gonocytes in comparison to their somatic populations. There were 24 common up-regulated genes found between the three groups. Mouse gonocytes shared 35% (44/125) of their differentially expressed genes with human prepubertal SSCs. In contrast, human prepubertal SSCs and mouse gonocytes shared 42% (443/1049) and 43% (54/125) of their differentially expressed genes, respectively, with SSEA-4+ cells. Alternatively, when only the top 50 upregulated genes from the human prepubertal SSCs and mouse gonocytes were evaluated, 68% (34/50) and 54% (27/50) of the differentially expressed genes, respectively, were in common with differentially upregulated genes seen in human testicular SSEA-4+ cells. Thus, the similarity between the transcriptome profiles of human testicular SSEA-4+ cells, human prepubertal SSCs, and mouse gonocytes further demonstrates that human adult SSCs are within the testicular SSEA-4+ population.

Developing the ability to isolate SSCs and understanding the testicular niche optimal for SSC growth and development are important to develop effective therapeutic options for pediatric cancer patients facing sterilizing treatments. Using confocal microscopy, FACS sorted subpopulations of testicular cells, time-lapse photography, comprehensive mRNA sequencing, and a novel in vitro culture system, the present invention identifies a subpopulation of adult human testicular cells highly enriched for SSCs and the support cells critical to their growth based on distinct extracellular markers SSEA-4 and THY1, respectively. These insights provide valuable information for the development of future treatments to preserve and restore fertility.

Both testicular THY1+ and SSEA-4+ cells have been reported to contain SSCs based on mouse and human studies. Specifically, transplantation of both enriched human testicular THY1+ and SSEA-4+ cells into mouse testes resulted in germ cell colony formation. Significant controversy suggests that neither in vitro culture of human unsorted testicular cells nor enriched THY1+ cells with the current system selected for SSC expansion. Rather, recent human studies suggest that the in vitro systems selected for cells of mesenchymal origin. The data described herein demonstrated that SSC colonies disappeared as THY1+ cells expanded in culture over time. Using confocal microscopy, the data described herein demonstrated that THY1+ cells were predominantly located in lamina propria with some expression on Sertoli cells. Sorted THY1+ cells did not express germ cell markers as evaluated by microscopy (VASA) or qPCR (DAZL and VASA) suggesting a somatic origin. In contrast, previous studies demonstrated that human THY1+ populations contain SSCs after mouse xenotransplantation. It is possible that this observation was the result of germ cell contamination given that an enriched rather than sorted cell population was analyzed. When sorted testicular THY1+ cells were used to establish mRNA profile by RNA sequencing, the THY1+ transcriptome was consistent with the many somatic cell types making up the seminiferous tubules. Additionally, the transcriptome profiles of the THY1+ and SSEA-4+ populations were quite distinct as shown in the PCA analysis. Further functional evidence that supports sorted testicular THY1+ cells as the population containing testicular multipotent stromal cells (TMS) include rapid binding to plastic, expression of consensus mesenchymal markers (CD73 and 105), and the ability to differentiate into adipocytes, chondrocytes, and osteocytes. Given the anatomic location of the THY1+ cells within the seminiferous tubules, the lack of germ cell markers, high expression of Vimentin, and the ability to differentiate into all mesenchymal lineages, the TMS (THY1+) population, demonstrated here, is of somatic origin and not germ cells.

SSEA-4 is a marker of undifferentiated pluripotent human embryonic stem cells, cleavage to blastocyst stage embryo, human fetal SSCs, and prepubertal SSCs. Anatomically, SSEA-4+ cells are located predominantly at the basement membrane suggesting that they are SSCs. This is in contrast to previous studies that demonstrated limited co-expression of THY1 and SSEA-4 on sub-populations of human testicular cells using conventional microscopy. However, these earlier studies did not utilize multicolor FACS or confocal microscopy analyses. As disclosed herein, multicolor FACS, confocal microscopy, and transcriptomes on sorted cells confirmed that THY1+ and SSEA-4+ cells were two distinct populations with different physical, cellular, and molecular profiles. When the tubules were stained for VASA, VASA bright (SSEA-4−) cells, contained haploid cells and located toward the lumen whereas the VASA dim (SSEA-4+) cells were found at the basement membrane. This finding is consistent with previous human studies demonstrating that high level of VASA expression was associated with maturing germ cells. In contrast to the absence of markers associated with meiosis (DMC1, SYCP3) or differentiating spermatids (PRM2, ACR), SSEA-4+ cells expressed high levels of putative SSC markers (ZBTB16, GFRA1, SALL4, MAGEA4, GPR125) and pluripotency genes (TERT and LIN28B), consistent with primitive spermatogonia containing SSCs. Lower levels of ZBTB16, GFRA1, and GPR125 expression was also detected in THY1+ cells in comparison to SSEA-4+ cells in these studies. This is similar to previous human and mouse studies in which ZBTB16, GFRA-1, and GPR125 are markers of SSCs but low level expression was also detected in testicular somatic cells. Of note, the genes (GFRA1, RET, ETV5) involved in the GDNF mediated SSC self-renewal were also highly expressed in the SSEA-4+ cells. These results support previous studies demonstrating germ cell colonization in mouse xenograft model following transplantation of primary SSEA-4+ sorted cells. Recent studies demonstrate that human bone marrow derived MSCs (BmMSCs) also express SSEA-4, but lack CD45 expression. Although CD45+ cells were excluded from any FACS analyses, the possibility of BmMSC contamination still exists. Perhaps, the small number of BmMSCs contributed to a very low level of VIM expression detected in the present SSEA-4+ population. However, sorted SSEA-4+ cells failed to survive in vitro in the absence of other cell types, suggesting that this possible contamination is very low at best.

The significant similarity in differentially expressed genes in human testicular SSEA-4+/THY1+ in comparison with human prepubertal SSCs/somatic cells and mouse gonocytes/somatic cells further solidifies that human adult SSCs reside with the SSEA-4+ population. Interestingly, of the 24 common upregulated genes between the three groups, 16 genes (ASF1B, ASPM, BUB1, CASC5, CENPA, CENPF, CENPO, EXO1, HELLS, KIF11, KNTC1, MCM8, RAD51AP1, RAD54B, STAG3, and TOP2A) are involved cell cycle, DNA replication, meiosis, and DNA repair regulations as demonstrated in the interactive pathway analysis. Three of the 24 common genes (DAZL, PIWIL4, and BNC1) are intricately involved with germ cell maintenance and fertility. When the top 50 differentially up-regulated genes from human prepubertal SSCs were compared with human testicular SSEA-4+ cells, 19 of the 34 commonly up-regulated genes (STK31, MAGEA4, TPTE, DDX4, TKTL1, MAEL, ELAVL2, LIN28B, TEX15, SNAP91, CENPE, SLC25A31, DPPA2, FGFR3, DPPA4, DAZL, CASC5, TOP2A, and CENPF) are also involved in pluripotency, cell cycle, meiosis, DNA repair, and germ cell regulations. Of the 16 non-overlapping genes, SOHLH2 and POLB were significantly up regulated in human SSEA-4+ population but at <log₂ 2-fold change. Of the 14 remaining non-overlapping genes, only one (CD109) has been well characterized. CD109 is well characterized in the hematopoietic system and its expression is likely due to contamination with the prepubertal SSC isolation as the SSCs were manually isolated. In contrast, when the top 50 up-regulated genes in the mouse gonocytes were compared with human testicular SSEA-4+ cells, 20 of the 27 commonly up-regulated genes (EPCAM, BNC1, CENPF, CDCA7L, DAZL, TOP2A, STAGS, MCM8, BUB1, RAD54B, KNTC1, EXO1, CASC5, PIWIL4, CDCA5, SALL4, RAD51, ERCC6L, TPX2, and CDCA2) are involved in the pluripotency, cell cycle, meiosis, DNA repair, and germ cell regulations. Thus, these findings highlight the evolutionary conserved genes in SSCs that are essential for reproduction.

When sorted THY1+, SSEA-4+, and THY1−/SSEA-4− cells were cultured separately on either uncoated or coated plates, only THY1+ cells adhered and grew in a monolayer fashion regardless of surface substrate. In contrast to the lack of germ cell expression, THY1+ cells continued to express THY1+/CD73+/CD105+ and VIMENTIN after more than 20 passages further confirming a mesenchymal origin. When unsorted testicular cells were cultured, germ cells quickly bound to the adherent cells, formed colonies within two weeks of culture, and continued to express SSEA-4 and VASA. However, the adherent cells quickly outgrew and subsequently inhibited the SSC colonies growth by 4 weeks of culture. To delineate the relationship between SSC growth and its required niche, sorted SSEA-4+ cells were cultured in the presence of irradiated MEFs, human placental fibroblasts, human fetal testicular fibroblasts, and sorted adult testicular THY1+ cells. SSEA-4+ cells bound exclusively to testicular THY1+ cells within 48 hours and established colony formations. Under these conditions, SSC colonies continued to expand in size and underwent many subsequent passages without loss of SSEA-4 and VASA expression. This observation is consistent with recent studies that have demonstrated the importance of testicular stromal cells as a source of essential growth factors required by SSCs. Previous studies demonstrated the presence of both SSCs and testis derived pluripotent stem cells when unsorted testicular cells were cultured in vivo. Thus far, this phenomenon has not been observed with either unsorted testicular cells or sorted testicular SSEA-4+ cells. All observed colonies in the present studies expressed SSEA-4 and VASA. Although the percentage of SSC colonies formed per sorted SSEA-4+ cells (25,000 cells per experiment) appeared to be low 0.02-0.1% using the systems of the present invention, the cell number required for engraftment of SSEA-4+ colonies using a mouse model was more than 300,000 cells/transplant. This suggests that the SSEA-4+ population is enriched for SSCs and only a small portion of the population can form colonies and repopulate in vivo. The number of cells germ cells needed for engraftment in autologous non-human primate transplant model was ˜100×10⁶ cells. Thus, the low initial concentration of SSCs in testicular tissue highlights the need for in vitro expansion of SSCs prior to developing clinically viable SSC transplantation techniques in the future. Even though MEFs were found to support in vitro mouse SSC expansion, this was not the case with human SSCs suggesting basic differences between human and mouse SSCs. These data provide strong evidence that SSEA-4 is a specific marker expressed in primitive SSCs while the somatic THY1+ cells are TMSCs that play an instrumental role in providing the appropriate niche required for SSC expansion.

SSEA-4+ cell xenotransplantation, prior to and after in vitro expansion, is not included here. Although allotransplantation of mouse SSCs into germ cell depleted testes is an ideal in vivo assay to evaluate for the ability of mouse SSCs to rescue spermatogenesis, it is suboptimal for human SSCs. Presumably due to interspecies differences, xenograft of human SSCs into mouse testes resulted in only colonization of human cells without differentiation. Furthermore, recent studies demonstrate that allogeneic MSC xenotransplantation also resulted in formation of germ cell colonies, highlighting this model's lack of specificity, especially in the absence of in vivo differentiation. Thus, there is no current ideal in vivo assay to evaluate human SSC activity.

Using a combination of multicolor FACS, confocal microscopy analyses, molecular profiling, and cell culture with sorted subpopulations of testicular cells monitored with time-lapse photography, the present invention provides methods and systems configured to definitively identify and differentiate the stromal and SSC compartments within the human testicular niche. In particular, the present invention identifies specific interactions between adult testicular THY1+ cells and SSCs that facilitate in vitro human SSC expansion. These data explain the controversy regarding MSC expansion with the traditional in vitro culture system using testicular cells.

Identifying and growing SSCs have paradigm-shifting implications for patients. To date, pre-pubertal males facing sterilizing chemotherapy do not have a proven means of protecting their fertility. In a small number of centers around the world, testicular biopsies are performed prior to chemotherapy in the hopes that new fertility restoring treatments will become available. Development of methods to expand purified SSCs exponentially, free of malignant cell contamination, for future autologous transplant or to differentiate pre-pubertal SSCs to mature sperm has enormous therapeutic potential. The present invention provides steps of isolating and expanding highly purified human SSCs using a defined somatic niche provided by the TMSCs. This is a critical step forward in developing strategies of prepubertal SSC expansion and autologous SSC therapy for fertility preservation treatments.

Further description of Example 1 is found in Smith et al., “Testicular Niche Required for Human Spermatogonial Stem Cell Expansion,” Stern Cells Trans Med September 2014 vol. 3 no. 9 1043-1054.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

TABLE II
Enriched genes in human testicular SSEA4+cells compared toThy1+ cells

| | Base | | Fold Change | log2 Fold | |
|---|---|---|---|---|---|
| | mean | Base mean | (SSEA-4+/THY- | change (SSEA- | |
| Genes | THY-1+ | SSEA-4+ | 1+) | 4+/Thy-1+) | P-value |
| SALL3 | 0.369841 | 265.799991 | 718.686406 | 9.489219 | 7.07E−25 |
| TCL1A | 0.369841 | 88.394342 | 239.006074 | 7.900903 | 1.10E−09 |
| GDA | 3.983499 | 888.982966 | 223.166373 | 7.801976 | 1.33E−46 |
| OR52E4 | 0.352453 | 72.112699 | 204.602442 | 7.676680 | 0.000267488 |
| LOC339166 | 0.727188 | 148.567204 | 204.303722 | 7.674572 | 7.86E−14 |
| TERT | 0.369841 | 69.047034 | 186.693628 | 7.544529 | 1.39E−07 |
| CHDC2 | 0.352453 | 61.203237 | 173.649466 | 7.440034 | 7.72E−07 |
| GJB2 | 1.097029 | 181.082991 | 165.066679 | 7.366905 | 3.06E−14 |
| PPP1R3A | 0.369841 | 61.005676 | 164.950908 | 7.365893 | 5.60E−07 |
| ITGAM | 0.369841 | 57.035875 | 154.217117 | 7.268819 | 0.047036405 |
| OTC | 0.357347 | 54.978263 | 153.851393 | 7.265394 | 0.000108254 |
| SLC17A2 | 0.369841 | 54.122635 | 146.340119 | 7.193182 | 3.40E−06 |
| ACTL8 | 3.685919 | 534.171190 | 144.922111 | 7.179134 | 3.92E−34 |
| NCOR1P1 | 1.084534 | 154.111639 | 142.099344 | 7.150756 | 1.04E−14 |
| RNF128 | 0.357347 | 50.174654 | 140.408956 | 7.133491 | 1.14E−05 |
| SLC39A12 | 0.352453 | 48.661235 | 138.064551 | 7.109199 | 2.18E−05 |
| B3GALT5 | 0.727188 | 97.852571 | 134.562972 | 7.072138 | 8.34E−10 |
| USH1C | 0.352453 | 46.981352 | 133.298287 | 7.058514 | 0.012480339 |
| HIST1H2BH | 0.369841 | 49.239924 | 133.137942 | 7.056778 | 0.000725555 |
| IRGM | 0.369841 | 48.274320 | 130.527083 | 7.028205 | 1.81E−05 |
| TAC4 | 0.352453 | 43.074927 | 122.214747 | 6.933275 | 8.52E−05 |
| SLC15A1 | 0.357347 | 42.946007 | 120.180280 | 6.909056 | 0.000838799 |
| PIWIL3 | 1.429386 | 170.283804 | 119.130722 | 6.896402 | 1.48E−15 |
| DGKK | 0.369841 | 41.731083 | 112.835076 | 6.818072 | 8.95E−05 |
| GPR114 | 1.109524 | 122.633363 | 110.527883 | 6.788267 | 9.72E−12 |
| BARHL2 | 0.727188 | 79.556240 | 109.402584 | 6.773503 | 3.18E−08 |
| CACNA1E | 2.206554 | 231.815806 | 105.057865 | 6.715040 | 9.85E−19 |
| CCR4 | 1.062252 | 110.108777 | 103.655975 | 6.695659 | 1.74E−10 |
| SOX1 | 1.466871 | 151.696632 | 103.415132 | 6.692303 | 5.30E−13 |

TABLE III
Enriched genes in human testicular Thy-1+ cells compared to SSEa-4+ cells.
Base mean expression values are expression values normalized as in Example 1.

| | | | Fold | | |
|---|---|---|---|---|---|
| | | | Change | log2 Fold | |
| | | | (SSEA- | change (SSE | |
| | Base mean | Base mean | 4+/THY- | A-4+/Thy- | |
| Gene | THY-1+ | SSEA-4+ | 1+) | 1+) | P-value |
| C8orf4 | 1552.281897 | 0.659752 | 0.000425 | −11.200179 | 4.02E−12 |
| KCNIP1 | 310.603343 | 0.209527 | 0.000675 | −10.533723 | 4.04E−29 |
| PTGDS | 126058.768173 | 103.768023 | 0.000823 | −10.246519 | 6.53E−79 |
| IGFBP7 | 34054.674055 | 28.909034 | 0.000849 | −10.202117 | 6.99E−113 |
| MEOX2 | 424.491659 | 0.362289 | 0.000853 | −10.194381 | 3.00E−36 |
| LOC100506990 | 230.579463 | 0.209527 | 0.000909 | −10.103914 | 8.68E−12 |
| BST2 | 1203.604467 | 1.199155 | 0.000996 | −9.971128 | 1.28E−17 |
| FLRT2 | 8654.047300 | 8.969451 | 0.001036 | −9.914140 | 4.01E−11 |
| MYC | 10099.202434 | 10.516014 | 0.001041 | −9.907438 | 2.40E−23 |
| C11orf96 | 9714.823779 | 10.306487 | 0.001061 | −9.880491 | 8.17E−100 |
| LUM | 6403.066902 | 7.578302 | 0.001184 | −9.722673 | 6.54E−17 |
| KRT13 | 1902.263106 | 2.286021 | 0.001202 | −9.700663 | 9.79E−11 |
| MT1M | 865.571599 | 1.047634 | 0.001210 | −9.690375 | 1.99E−38 |
| PCDHB5 | 172.842048 | 0.209527 | 0.001212 | −9.688104 | 2.34E−09 |
| CAPN6 | 999.757137 | 1.231567 | 0.001232 | −9.664938 | 7.37E−29 |
| SPARCL1 | 39920.002334 | 49.53644 | 0.001241 | −9.654406 | 4.25E−32 |
| CX3CL1 | 1659.859450 | 2.095267 | 0.001262 | −9.629711 | 1.06E−29 |
| C7 | 10136.252236 | 13.01589 | 0.001284 | −9.605034 | 1.58E−28 |
| DCN | 46901.616536 | 60.37922 | 0.001287 | −9.601370 | 5.85E−12 |
| MFAP4 | 6083.235754 | 8.051638 | 0.001324 | −9.561341 | 1.70E−91 |
| ENG | 14987.486673 | 20.66832 | 0.001379 | −9.502121 | 1.02E−45 |
| CYR61 | 22458.070506 | 31.54307 | 0.001405 | −9.475695 | 4.95E−56 |
| ATP1A2 | 1233.469655 | 1.835796 | 0.001488 | −9.392101 | 3.95E−43 |
| APOD | 13160.979847 | 20.523967 | 0.001559 | −9.324742 | 1.77E−76 |
| RARRES2 | 564.556015 | 0.901691 | 0.001597 | −9.290267 | 9.10E−29 |
| IFI44 | 1875.879401 | 3.043011 | 0.001622 | −9.267852 | 3.41E−22 |
| ROBO4 | 629.464558 | 1.022041 | 0.001624 | −9.266529 | 0.000654098 |
| SELENBP1 | 507.744100 | 0.838107 | 0.001651 | −9.242751 | 7.36E−08 |
| TM4SF1 | 15192.272407 | 25.710779 | 0.001692 | −9.206749 | 3.89E−14 |
| BGN | 10520.133109 | 18.66674 | 0.001774 | −9.138467 | 2.43E−34 |
| PCDHB4 | 115.708963 | 0.209527 | 0.001811 | −9.109151 | 3.16E−10 |
| SFRP4 | 3524.078576 | 6.508968 | 0.001847 | −9.080601 | 2.51E−59 |
| IFITM1 | 2520.876088 | 4.749155 | 0.001884 | −9.052039 | 1.83E−47 |
| DPEP1 | 1607.059571 | 3.028131 | 0.001884 | −9.051780 | 5.69E−26 |
| PROK1 | 5115.354612 | 10.237939 | 0.002001 | −8.964765 | 2.52E−85 |
| MIR221 | 104.536361 | 0.209527 | 0.002004 | −8.962655 | 1.74E−05 |
| TMEM119 | 540.816418 | 1.086866 | 0.002010 | −8.958821 | 0.000552842 |
| IGF1 | 14713.605539 | 30.395736 | 0.002066 | −8.919066 | 5.85E−06 |
| TMEM173 | 1560.957773 | 3.284950 | 0.002104 | −8.892344 | 2.98E−64 |
| MGP | 12700.277122 | 26.727241 | 0.002104 | −8.892333 | 2.38E−44 |
| THBD | 9383.653465 | 19.947644 | 0.002126 | −8.877788 | 4.61E−51 |
| GGT5 | 351.942848 | 0.748929 | 0.002128 | −8.876296 | 4.77E−12 |
| SERPINE1 | 10975.522501 | 23.384912 | 0.002131 | −8.874496 | 5.90E−89 |
| ESAM | 783.538402 | 1.683034 | 0.002148 | −8.862796 | 2.76E−05 |
| HSPB6 | 1467.506276 | 3.188953 | 0.002173 | −8.846068 | 6.00E−23 |
| CCL2 | 8562.070816 | 18.706775 | 0.002185 | −8.838255 | 4.09E−26 |
| MIR100HG | 1787.017577 | 3.969054 | 0.002221 | −8.814543 | 3.87E−53 |
| CREB3L1 | 445.502056 | 0.990869 | 0.002224 | −8.812522 | 3.60E−08 |
| MTMR9LP | 336.559917 | 0.748929 | 0.002225 | −8.811818 | 5.40E−29 |
| PCDHGC3 | 475.021797 | 1.086866 | 0.002288 | −8.771676 | 4.34E−37 |
| LHX9 | 684.496667 | 1.626269 | 0.002376 | −8.717334 | 6.52E−13 |
| LOC100132891 | 1104.727283 | 2.634670 | 0.002385 | −8.711852 | 5.64E−39 |
| IFITM3 | 6857.103610 | 16.612748 | 0.002423 | −8.689165 | 9.12E−48 |
| EPDR1 | 1658.205446 | 4.027060 | 0.002429 | −8.685680 | 5.11E−63 |
| HCG26 | 85.544094 | 0.209527 | 0.002449 | −8.673390 | 0.001262088 |
| ADRA2A | 752.164313 | 1.860148 | 0.002473 | −8.659487 | 6.51E−46 |
| INHBA | 508.661998 | 1.263980 | 0.002485 | −8.652590 | 0.006779993 |
| ECSCR | 247.496215 | 0.628580 | 0.002540 | −8.621094 | 4.15E−05 |
| ISLR | 2557.852612 | 6.540140 | 0.002557 | −8.611396 | 7.98E−70 |
| TCF23 | 139.263960 | 0.362289 | 0.002601 | −8.586466 | 2.98E−15 |
| EMP1 | 16956.072099 | 44.47200 | 0.002623 | −8.574689 | 3.74E−40 |
| DPT | 1786.468248 | 4.758457 | 0.002664 | −8.552401 | 3.97E−31 |
| PCDHB11 | 123.241988 | 0.329876 | 0.002677 | −8.545354 | 1.15E−13 |
| CDKN1A | 23872.675006 | 64.837919 | 0.002716 | −8.524307 | 1.43E−30 |
| IGFN1 | 1561.356100 | 4.265276 | 0.002732 | −8.515945 | 1.79E−52 |
| CD248 | 919.248401 | 2.560373 | 0.002785 | −8.487957 | 2.03E−19 |
| LCN6 | 117.944658 | 0.329876 | 0.002797 | −8.481971 | 0.001310463 |
| BHLHE40 | 18325.305707 | 52.142595 | 0.002845 | −8.457159 | 8.11E−14 |
| IL6 | 5826.562479 | 16.590878 | 0.002847 | −8.456111 | 2.13E−32 |
| ZFP36 | 86439.323100 | 246.656043 | 0.002854 | −8.453043 | 5.27E−92 |
| CDH5 | 1633.674271 | 4.670521 | 0.002859 | −8.450321 | 9.85E−06 |
| PCDHB12 | 123.360685 | 0.362289 | 0.002937 | −8.411527 | 1.09E−13 |
| EGR3 | 17627.076947 | 51.942188 | 0.002947 | −8.406671 | 3.44E−39 |
| ADAMTS1 | 23601.751507 | 70.174385 | 0.002973 | −8.393734 | 9.27E−17 |
| CYBRD1 | 13050.657588 | 39.699400 | 0.003042 | −8.360790 | 3.04E−86 |
| CFH | 6471.084351 | 20.330391 | 0.003142 | −8.314226 | 1.98E−18 |
| G0S2 | 458.199420 | 1.441094 | 0.003145 | −8.312667 | 8.18E−12 |
| COL15A1 | 19371.930457 | 61.531648 | 0.003176 | −8.298423 | 1.56E−36 |
| PLAC9 | 583.527537 | 1.891319 | 0.003241 | −8.269264 | 6.02E−40 |
| CBLN4 | 431.872807 | 1.409922 | 0.003265 | −8.258847 | 9.38E−20 |
| GADD45G | 1048.346428 | 3.437712 | 0.003279 | −8.252451 | 4.37E−53 |
| TNFRSF14 | 229.270320 | 0.781342 | 0.003408 | −8.196879 | 5.72E−18 |
| HOXD4 | 61.022970 | 0.209527 | 0.003434 | −8.186074 | 4.09E−07 |
| JUNB | 30780.450532 | 106.954232 | 0.003475 | −8.168877 | 2.26E−67 |
| MSC | 630.024838 | 2.190024 | 0.003476 | −8.168319 | 1.19E−40 |
| PCDHGA6 | 103.108238 | 0.362289 | 0.003514 | −8.152804 | 1.22E−11 |
| LTBP4 | 6243.358695 | 22.028475 | 0.003528 | −8.146809 | 3.04E−79 |
| FLI1-AS1 | 58.320371 | 0.209527 | 0.003593 | −8.120722 | 0.000756805 |
| PCDHB14 | 200.940736 | 0.724577 | 0.003606 | −8.115415 | 7.11E−20 |
| PCDHB9 | 57.681492 | 0.209527 | 0.003632 | −8.104830 | 9.92E−07 |
| PODN | 3263.613463 | 11.908234 | 0.003649 | −8.098367 | 9.43E−73 |
| PRRX1 | 9393.052618 | 34.685991 | 0.003693 | −8.081097 | 3.83E−14 |
| S100A16 | 422.300129 | 1.561443 | 0.003697 | −8.079245 | 2.05E−09 |
| PLA1A | 462.575367 | 1.715446 | 0.003708 | −8.074961 | 1.26E−07 |
| ENPP6 | 883.916771 | 3.334895 | 0.003773 | −8.050125 | 1.53E−40 |
| COL1A2 | 12228.696886 | 46.215973 | 0.003779 | −8.047663 | 2.64E−14 |
| PRELP | 2420.882822 | 9.233260 | 0.003814 | −8.034477 | 6.84E−13 |
| SERPINA3 | 1264.853574 | 4.904399 | 0.003877 | −8.010678 | 1.69E−07 |
| KCNMB1 | 546.964584 | 2.126439 | 0.003888 | −8.006864 | 1.90E−36 |
| IL32 | 370.354715 | 1.449155 | 0.003913 | −7.997552 | 7.15E−06 |
| APLNR | 396.452643 | 1.562684 | 0.003942 | −7.986978 | 0.003749569 |
| TMEM204 | 228.648404 | 0.901691 | 0.003944 | −7.986281 | 1.99E−21 |
| SELE | 28817.453144 | 117.039176 | 0.004061 | −7.943808 | 0.008625091 |
| PTGS2 | 6485.233709 | 26.380629 | 0.004068 | −7.941536 | 3.44E−15 |
| TNS1 | 23491.647934 | 96.072523 | 0.004090 | −7.933808 | 4.17E−84 |
| WFDC1 | 773.718874 | 3.206486 | 0.004144 | −7.914673 | 8.08E−43 |
| SOCS3 | 16393.694577 | 68.644886 | 0.004187 | −7.899773 | 1.88E−33 |
| LGALS3BP | 2232.883618 | 9.575090 | 0.004288 | −7.865406 | 9.04E−63 |
| ATF3 | 10763.344980 | 46.165139 | 0.004289 | −7.865107 | 1.51E−77 |
| ACTA2 | 22090.973395 | 95.712740 | 0.004333 | −7.850530 | 4.64E−81 |
| CILP | 3975.257563 | 17.956264 | 0.004517 | −7.790417 | 0.000702403 |
| BTG2 | 36331.831024 | 165.554024 | 0.004557 | −7.777788 | 7.95E−82 |
| INMT | 1454.885818 | 6.657837 | 0.004576 | −7.771637 | 1.02E−52 |
| CXCL1 | 502.723579 | 2.304794 | 0.004585 | −7.768984 | 0.000289919 |
| EGR1 | 229062.079514 | 1064.660282 | 0.004648 | −7.749202 | 2.27E−83 |
| ICAM1 | 14114.383384 | 65.638491 | 0.004650 | −7.748408 | 2.84E−17 |
| TBX2 | 313.291432 | 1.473507 | 0.004703 | −7.732108 | 3.12E−21 |
| S100A6 | 2610.596420 | 12.549552 | 0.004807 | −7.700600 | 4.20E−65 |
| PEAR1 | 1707.339649 | 8.285347 | 0.004853 | −7.686972 | 3.86E−34 |
| CCRL2 | 171.658571 | 0.838107 | 0.004882 | −7.678192 | 0.013001853 |
| OSR1 | 846.093611 | 4.173002 | 0.004932 | −7.663588 | 1.04E−43 |
| LURAP1L | 42.473430 | 0.209527 | 0.004933 | −7.663282 | 4.97E−05 |
| CLEC3B | 1138.374771 | 5.703274 | 0.005010 | −7.640970 | 4.12E−05 |
| PCDHB16 | 237.548238 | 1.200396 | 0.005053 | −7.628567 | 8.19E−21 |
| NUPR1 | 1240.070773 | 6.330613 | 0.005105 | −7.613861 | 5.16E−36 |
| MFAP5 | 1773.806582 | 9.077402 | 0.005117 | −7.610354 | 0.002043518 |
| ELN | 443.649570 | 2.287262 | 0.005156 | −7.599655 | 5.65E−32 |
| MT1A | 3325.842597 | 17.156659 | 0.005159 | −7.598807 | 1.77E−17 |
| LOC100506795 | 40.468776 | 0.209527 | 0.005177 | −7.593531 | 0.000363834 |
| CAV2 | 1572.407024 | 8.174469 | 0.005199 | −7.587634 | 1.73E−48 |
| CYP1B1 | 4752.793901 | 24.855140 | 0.005230 | −7.579088 | 2.89E−50 |
| HLA-E | 9842.815330 | 52.394450 | 0.005323 | −7.553513 | 4.66E−52 |
| LCNL1 | 67.565586 | 0.362289 | 0.005362 | −7.543005 | 7.92E−08 |
| TIMP3 | 46159.853231 | 247.873541 | 0.005370 | −7.540891 | 2.63E−80 |
| EMX2 | 349.002359 | 1.956145 | 0.005605 | −7.479080 | 8.13E−28 |
| XAF1 | 2138.375276 | 12.496511 | 0.005844 | −7.418846 | 2.60E−59 |
| CDH16 | 106.925444 | 0.628580 | 0.005879 | −7.410293 | 1.27E−08 |
| PLAC1 | 91.668153 | 0.539403 | 0.005884 | −7.408914 | 8.74E−10 |
| FLI1 | 654.320149 | 3.931063 | 0.006008 | −7.378933 | 0.000216206 |
| TINAGL1 | 179.982709 | 1.086866 | 0.006039 | −7.371540 | 7.56E−05 |
| SLC16A2 | 907.564244 | 5.485687 | 0.006044 | −7.370184 | 2.90E−43 |
| PPAP2B | 10454.263487 | 63.484135 | 0.006073 | −7.363480 | 4.36E−10 |
| PLP2 | 1171.409303 | 7.137549 | 0.006093 | −7.358601 | 1.27E−39 |
| NID1 | 5394.460226 | 32.908920 | 0.006101 | −7.356856 | 1.74E−34 |
| PCDH18 | 4318.408006 | 26.568274 | 0.006152 | −7.344651 | 5.67E−67 |
| ARX | 1271.608551 | 7.847690 | 0.006171 | −7.340171 | 9.78E−52 |
| ANXA1 | 6030.152088 | 38.000245 | 0.006302 | −7.310042 | 1.08E−10 |
| RGN | 550.084970 | 3.472777 | 0.006313 | −7.307421 | 6.92E−08 |
| PNP | 3077.210125 | 19.471826 | 0.006328 | −7.304091 | 6.30E−06 |
| HS3ST2 | 83.610147 | 0.539403 | 0.006451 | −7.276171 | 5.57E−09 |
| GIMAP7 | 170.760007 | 1.111218 | 0.006507 | −7.263684 | 1.07E−06 |
| ANG | 109.945214 | 0.724577 | 0.006590 | −7.245429 | 5.84E−07 |
| MAOB | 1347.767920 | 8.915338 | 0.006615 | −7.240067 | 5.51E−50 |
| ETS2 | 5904.246772 | 39.085426 | 0.006620 | −7.238978 | 1.12E−07 |
| RASSF2 | 5015.653938 | 33.207964 | 0.006621 | −7.238765 | 4.63E−67 |
| LOC100507412 | 239.640544 | 1.626269 | 0.006786 | −7.203162 | 3.34E−11 |
| WISP2 | 496.259963 | 3.420350 | 0.006892 | −7.180808 | 2.65E−06 |
| CPZ | 163.331587 | 1.143631 | 0.007002 | −7.158039 | 0.001372574 |
| FOS | 105750.801451 | 741.444221 | 0.007011 | −7.156115 | 2.17E−75 |
| TSHZ2 | 5676.111216 | 40.086942 | 0.007062 | −7.145627 | 1.49E−67 |
| PLA2G5 | 805.308312 | 5.740024 | 0.007128 | −7.132341 | 8.47E−05 |
| FHL5 | 1891.285014 | 13.493755 | 0.007135 | −7.130931 | 6.20E−53 |
| APOLD1 | 2430.072308 | 17.411623 | 0.007165 | −7.124805 | 0.000612391 |
| ST6GALNAC1 | 200.773358 | 1.449155 | 0.007218 | −7.114212 | 0.004539169 |
| GPRC5A | 1714.377014 | 12.406889 | 0.007237 | −7.110399 | 4.18E−51 |
| RBMS3 | 4142.744648 | 29.998382 | 0.007241 | −7.109558 | 9.90E−64 |
| MIR604 | 99.811223 | 0.724577 | 0.007259 | −7.105918 | 2.09E−07 |
| COL1A1 | 5171.249842 | 37.903451 | 0.007330 | −7.092040 | 8.65E−11 |
| SNAI2 | 1966.706771 | 14.604176 | 0.007426 | −7.073257 | 2.39E−40 |
| SMOC2 | 7850.320663 | 59.606559 | 0.007593 | −7.041137 | 1.24E−68 |
| PCDHGA5 | 75.127554 | 0.571815 | 0.007611 | −7.037649 | 0.001148967 |
| TCEAL7 | 212.270989 | 1.618208 | 0.007623 | −7.035366 | 1.12E−06 |
| OSR2 | 9161.084159 | 70.256116 | 0.007669 | −7.026751 | 1.08E−58 |
| DDR2 | 1185.861927 | 9.123454 | 0.007694 | −7.022140 | 8.14E−48 |
| CRYAB | 1648.226315 | 12.691157 | 0.007700 | −7.020947 | 1.76E−51 |
| MEG3 | 8787.447935 | 68.371605 | 0.007781 | −7.005903 | 7.18E−07 |
| MYCT1 | 929.830143 | 7.245944 | 0.007793 | −7.003650 | 0.000799888 |
| KLF2 | 6300.795879 | 49.208752 | 0.007810 | −7.000475 | 4.53E−22 |
| VIM | 37021.987597 | 290.863795 | 0.007857 | −6.991895 | 1.94E−18 |
| SEMA3D | 2843.495413 | 22.341445 | 0.007857 | −6.991799 | 1.80E−31 |
| LY6G6C | 53.305238 | 0.419053 | 0.007861 | −6.990999 | 9.00E−06 |
| 37316 | 485.959720 | 3.840644 | 0.007903 | −6.983345 | 9.09E−12 |
| HLA-C | 2656.018283 | 21.066753 | 0.007932 | −6.978153 | 2.31E−09 |
| FBLN5 | 13619.822444 | 108.810528 | 0.007989 | −6.967746 | 3.71E−39 |
| MAFB | 1559.716384 | 12.505812 | 0.008018 | −6.962541 | 4.47E−51 |
| PALMD | 960.712506 | 7.724244 | 0.008040 | −6.958567 | 2.20E−10 |
| FGF10 | 70.965592 | 0.571815 | 0.008058 | −6.955426 | 0.00047562 |
| SAT1 | 15733.721805 | 127.561682 | 0.008108 | −6.946521 | 2.37E−68 |
| TCEAL3 | 674.607903 | 5.498085 | 0.008150 | −6.938976 | 1.43E−37 |
| PI16 | 1901.020800 | 15.512687 | 0.008160 | −6.937182 | 7.05E−11 |
| IFI6 | 1912.300033 | 15.897643 | 0.008313 | −6.910352 | 2.32E−35 |
| GPNMB | 6751.265957 | 56.254038 | 0.008332 | −6.907058 | 1.17E−63 |
| CPE | 5989.057141 | 50.043434 | 0.008356 | −6.903004 | 2.63E−25 |
| PCDHGA2 | 143.032971 | 1.200396 | 0.008392 | −6.896694 | 9.40E−08 |
| PFN1P2 | 24.906706 | 0.209527 | 0.008412 | −6.893256 | 0.004987194 |
| FAM107A | 1199.549589 | 10.153555 | 0.008464 | −6.884364 | 1.87E−06 |
| NEDD9 | 1926.697697 | 16.331406 | 0.008476 | −6.882337 | 2.98E−09 |
| HSPA6 | 649.204500 | 5.539799 | 0.008533 | −6.872695 | 4.04E−06 |
| MYLIP | 5233.427358 | 44.746189 | 0.008550 | −6.869847 | 5.18E−15 |
| C1S | 11899.261599 | 102.712932 | 0.008632 | −6.856110 | 2.03E−14 |
| TIMP1 | 5699.304817 | 49.546244 | 0.008693 | −6.845866 | 2.01E−49 |
| WWTR1 | 4900.873832 | 42.953793 | 0.008765 | −6.834110 | 1.95E−31 |
| STEAP4 | 3940.412104 | 34.782877 | 0.008827 | −6.823825 | 1.79E−15 |
| ADIRF | 430.125147 | 3.808232 | 0.008854 | −6.819491 | 1.63E−28 |
| ADAMTS4 | 2374.718061 | 21.101361 | 0.008886 | −6.814276 | 4.90E−17 |
| MEG8 | 97.542614 | 0.869279 | 0.008912 | −6.810070 | 7.47E−10 |
| ACP5 | 69.813049 | 0.628580 | 0.009004 | −6.795256 | 4.29E−07 |
| NR4A2 | 10254.890716 | 92.610142 | 0.009031 | −6.790926 | 2.86E−33 |
| B2M | 20327.059518 | 183.800094 | 0.009042 | −6.789120 | 3.11E−13 |
| H19 | 3027.447425 | 27.413815 | 0.009055 | −6.787055 | 1.47E−08 |
| FOSL1 | 1241.824942 | 11.262461 | 0.009069 | −6.784796 | 2.25E−14 |
| SNORD114- | 36.367306 | 0.329876 | 0.009071 | −6.784575 | 0.000248541 |
| 12 | | | | | |
| TCF21 | 707.746986 | 6.427851 | 0.009082 | −6.782753 | 3.27E−11 |
| HTRA1 | 1237.830897 | 11.421428 | 0.009227 | −6.759927 | 2.28E−45 |
| FXYD1 | 119.893952 | 1.111218 | 0.009268 | −6.753473 | 1.58E−11 |
| PCDHB19P | 113.750018 | 1.054453 | 0.009270 | −6.753228 | 1.33E−05 |
| A4GALT | 128.217552 | 1.200396 | 0.009362 | −6.738940 | 1.00E−06 |
| LGALS1 | 4121.555770 | 38.611177 | 0.009368 | −6.738027 | 1.52E−48 |
| APOL1 | 532.426027 | 4.993577 | 0.009379 | −6.736364 | 7.56E−32 |
| S100A4 | 247.790105 | 2.342786 | 0.009455 | −6.724750 | 4.91E−20 |
| MYL9 | 4482.469099 | 42.477074 | 0.009476 | −6.721465 | 1.94E−31 |
| FOSB | 128099.394837 | 1217.816353 | 0.009507 | −6.716823 | 2.42E−67 |
| LOC100507463 | 81.333723 | 0.781342 | 0.009607 | −6.701755 | 6.13E−05 |
| JUN | 40161.152524 | 387.280450 | 0.009643 | −6.696278 | 1.24E−68 |
| LY96 | 21.609682 | 0.209527 | 0.009696 | −6.688400 | 0.011493175 |
| LRRN4CL | 194.291389 | 1.892560 | 0.009741 | −6.681739 | 1.61E−16 |
| MRGPRF | 304.383074 | 2.979426 | 0.009788 | −6.674710 | 4.82E−23 |
| CISH | 721.110248 | 7.100002 | 0.009846 | −6.666257 | 1.01E−17 |
| PDGFRB | 6256.083994 | 61.876574 | 0.009891 | −6.659723 | 2.67E−49 |
| C6 | 1430.033026 | 14.236139 | 0.009955 | −6.650347 | 0.00537999 |
| LOC100505865 | 21.023576 | 0.209527 | 0.009966 | −6.648730 | 0.013029223 |
| SNORD114-7 | 20.972145 | 0.209527 | 0.009991 | −6.645196 | 0.013642234 |
| SALL3 | 0.369841 | 265.799991 | 718.686406 | 9.489219 | 7.07E−25 |
| TCL1A | 0.369841 | 88.394342 | 239.006074 | 7.900903 | 1.10E−09 |
| GDA | 3.983499 | 888.982966 | 223.166373 | 7.801976 | 1.33E−46 |
| OR52E4 | 0.352453 | 72.112699 | 204.602442 | 7.676680 | 0.000267488 |
| LOC339166 | 0.727188 | 148.567204 | 204.303722 | 7.674572 | 7.86E−14 |
| TERT | 0.369841 | 69.047034 | 186.693628 | 7.544529 | 1.39E−07 |
| CHDC2 | 0.352453 | 61.203237 | 173.649466 | 7.440034 | 7.72E−07 |
| GJB2 | 1.097029 | 181.082991 | 165.066679 | 7.366905 | 3.06E−14 |
| PPP1R3A | 0.369841 | 61.005676 | 164.950908 | 7.365893 | 5.60E−07 |
| ITGAM | 0.369841 | 57.035875 | 154.217117 | 7.268819 | 0.047036405 |
| OTC | 0.357347 | 54.978263 | 153.851393 | 7.265394 | 0.000108254 |
| SLC17A2 | 0.369841 | 54.122635 | 146.340119 | 7.193182 | 3.40E−06 |
| ACTL8 | 3.685919 | 534.171190 | 144.922111 | 7.179134 | 3.92E−34 |
| NCOR1P1 | 1.084534 | 154.111639 | 142.099344 | 7.150756 | 1.04E−14 |
| RNF128 | 0.357347 | 50.174654 | 140.408956 | 7.133491 | 1.14E−05 |
| SLC39A12 | 0.352453 | 48.661235 | 138.064551 | 7.109199 | 2.18E−05 |
| B3GALT5 | 0.727188 | 97.852571 | 134.562972 | 7.072138 | 8.34E−10 |
| USH1C | 0.352453 | 46.981352 | 133.298287 | 7.058514 | 0.012480339 |
| HIST1H2BH | 0.369841 | 49.239924 | 133.137942 | 7.056778 | 0.000725555 |
| IRGM | 0.369841 | 48.274320 | 130.527083 | 7.028205 | 1.81E−05 |
| TAC4 | 0.352453 | 43.074927 | 122.214747 | 6.933275 | 8.52E−05 |
| SLC15A1 | 0.357347 | 42.946007 | 120.180280 | 6.909056 | 0.000838799 |
| PIWIL3 | 1.429386 | 170.283804 | 119.130722 | 6.896402 | 1.48E−15 |
| DGKK | 0.369841 | 41.731083 | 112.835076 | 6.818072 | 8.95E−05 |
| GPR114 | 1.109524 | 122.633363 | 110.527883 | 6.788267 | 9.72E−12 |
| BARHL2 | 0.727188 | 79.556240 | 109.402584 | 6.773503 | 3.18E−08 |
| CACNA1E | 2.206554 | 231.815806 | 105.057865 | 6.715040 | 9.85E−19 |
| CCR4 | 1.062252 | 110.108777 | 103.655975 | 6.695659 | 1.74E−10 |
| SOX1 | 1.466871 | 151.696632 | 103.415132 | 6.692303 | 5.30E−13 |

TABLE 4
Common differentially expressed genes in human adult SSCs (labeled as SSEA-4+
in this table), human prepubertal SSCs, and mouse gonocytes
Adult Human SSC's

| | | log2 Fold | |
|---|---|---|---|
| | | change (SSEA- | |
| Genes | Gene Description | 4+/Thy-1+) | P-value |
| CDCA7L | cell division cycle associated 7- | 4.501745647 | 1.02E−23 |
| | like | | |
| CENPA | centromere protein A | 4.16141603 | 8.08E−07 |
| CASC5 | caner susceptibility candidate 5 | 4.0149258 | 6.67E−31 |
| ASPM | abnormal spindle-like | 3.863320085 | 1.02E−23 |
| | microcephaly-assoicated protein | | |
| ASF1B | anti-silencing function 1B | 3.671074116 | 2.56E−08 |
| | histone chaperone | | |
| DAZL | deleted in azoospermia-like | 3.603712266 | 4.34E−25 |
| PIWIL4 | piwi-like RNA-mediated gene | 3.479879916 | 4.45E−21 |
| | silencing 4 | | |
| KIF11 | kinesin family member 11 | 3.397707376 | 7.73E−18 |
| RIMKLA | ribosomal modification protein | 3.214710751 | 2.88E−10 |
| | rimK-like family member A | | |
| BUB1 | BUB1 mitotic checkpoint | 3.197497471 | 1.22E−16 |
| | serine/threonine kinase | | |
| CENPO | centromere protein O | 3.183801598 | 1.50E−13 |
| EXO1 | exonuclease 1 | 3.141152487 | 2.67E−08 |
| CENPF | centromere protein F, | 2.998205177 | 1.06E−18 |
| | 350/400 kDA | | |
| HELLS | helicase, lymphoid-specific | 2.843209037 | 9.87E−12 |
| MCM8 | minichromosome maintenance | 2.798540593 | 1.06E−13 |
| | complex component 8 | | |
| TOP2A | topoisomerase (DNA) II alpha | 2.702211399 | 8.13E−14 |
| | 170 kDA | | |
| ADAL | adenosine deaminase-like | 2.608616705 | 1.52E−09 |
| KNTC1 | kinetochore associated 1 | 2.461552637 | 1.99E−13 |
| SHCBP1 | SHC SH2-domain binding | 2.431023277 | 4.01E−05 |
| | protein 1 | | |
| BNC1 | basonuclin 1 | 2.393465777 | 2.01E−05 |
| RAD54B | RAD54 homolog B | 2.145521887 | 0.003329563 |
| EPCAM | epithelial cell adhesion molecule | 2.071847523 | 2.09E−06 |
| RAD51AP1 | RAD51 associated protein 1 | 2.042633282 | 0.001055539 |
| STAG3 | stromal antigen 3 | 2.008151387 | 2.22E−06 |
| CXCL2 | chemokine (C—X—C motif) | −5.980122041 | 8.84E−07 |
| | ligan 2 | | |

Human Prepubertal SSCs

| | | log2 Fold | |
|---|---|---|---|
| | | change (SSC's/ | |
| Genes | Gene Description | Somatics) | P-value |
| CDCA7L | cell division cycle associated 7- | 3.546779321 | 0.013341062 |
| | like | | |
| CENPA | centromere protein A | 4.608017387 | 0.004369633 |
| CASC5 | caner susceptibility candidate 5 | 6.104959245 | 0.000896096 |
| ASPM | abnormal spindle-like | 3.684413293 | 0.005904672 |
| | microcephaly-assoicated protein | | |
| ASF1B | anti-silencing function 1B | 2.511272375 | 0.049124313 |
| | histone chaperone | | |
| DAZL | deleted in azoospermia-like | 6.59791986 | 0.00075621 |
| PIWIL4 | piwi-like RNA-mediated gene | 3.546129673 | 0.046719729 |
| | silencing 4 | | |
| KIF11 | kinesin family member 11 | 4.674878981 | 0.00226069 |
| RIMKLA | ribosomal modification protein | 3.516748237 | 0.002387756 |
| | rimK-like family member A | | |
| BUB1 | BUB1 mitotic checkpoint | 3.275292785 | 0.006676465 |
| | serine/threonine kinase | | |
| CENPO | centromere protein O | 3.930547774 | 0.039080744 |
| EXO1 | exonuclease 1 | 2.765250987 | 0.013774717 |
| CENPF | centromere protein F, | 5.620123527 | 0.003537705 |
| | 350/400 kDA | | |
| HELLS | helicase, lymphoid-specific | 4.668064373 | 0.004223187 |
| MCM8 | minichromosome maintenance | 4.707954449 | 0.005598506 |
| | complex component 8 | | |
| TOP2A | topoisomerase (DNA) II alpha | 5.73269873 | 0.001281089 |
| | 170 kDA | | |
| ADAL | adenosine deaminase-like | 3.470458487 | 0.005181568 |
| KNTC1 | kinetochore associated 1 | 4.872622483 | 0.003910136 |
| SHCBP1 | SHC SH2-domain binding | 3.350589677 | 0.006515753 |
| | protein 1 | | |
| BNC1 | basonuclin 1 | 3.440776308 | 0.007170407 |
| RAD54B | RAD54 homolog B | 4.150800058 | 0.005103767 |
| EPCAM | epithelial cell adhesion molecule | 2.015561271 | 0.044789063 |
| RAD51AP1 | RAD51 associated protein 1 | 4.41866676 | 0.005127404 |
| STAG3 | stromal antigen 3 | 3.637889447 | 0.030099148 |
| CXCL2 | chemokine (C—X—C motif) | −4.602867229 | 0.016693041 |
| | ligan 2 | | |

Mouse Gonocytes

| | | log2 Fold | |
|---|---|---|---|
| | | change (gonocytes/ | |
| Genes | Gene Description | somatic) | P-value |
| CDCA7L | cell division cycle associated 7- | 5.466068235 | 0.045136159 |
| | like | | |
| CENPA | centromere protein A | 3.165486378 | 0.033803261 |
| CASC5 | caner susceptibility candidate 5 | 4.33669684 | 0.045245424 |
| ASPM | abnormal spindle-like | 2.955299705 | 0.041020993 |
| | microcephaly-assoicated protein | | |
| ASF1B | anti-silencing function 1B | 3.527483812 | 0.045245424 |
| | histone chaperone | | |
| DAZL | deleted in azoospermia-like | 5.20237215 | 0.044830203 |
| PIWIL4 | piwi-like RNA-mediated gene | 4.335284608 | 0.013059441 |
| | silencing 4 | | |
| KIF11 | kinesin family member 11 | 3.967609178 | 0.03276366 |
| RIMKLA | ribosomal modification protein | 3.154493004 | 0.023451651 |
| | rimK-like family member A | | |
| BUB1 | BUB1 mitotic checkpoint | 4.697495567 | 0.012218183 |
| | serine/threonine kinase | | |
| CENPO | centromere protein O | 2.169229608 | 0.045245424 |
| EXO1 | exonuclease 1 | 4.496151535 | 0.012218183 |
| CENPF | centromere protein F, | 5.510356388 | 0.012218183 |
| | 350/400 kDA | | |
| HELLS | helicase, lymphoid-specific | 4.121928463 | 0.033803261 |
| MCM8 | minichromosome maintenance | 4.831815768 | 0.017412766 |
| | complex component 8 | | |
| TOP2A | topoisomerase (DNA) II alpha | 4.85171977 | 0.021153001 |
| | 170 kDA | | |
| ADAL | adenosine deaminase-like | 3.421623642 | 0.031405917 |
| KNTC1 | kinetochore associated 1 | 4.498854669 | 0.024039919 |
| SHCBP1 | SHC SH2-domain binding | 3.865462799 | 0.012218183 |
| | protein 1 | | |
| BNC1 | basonuclin 1 | 5.546043758 | 0.045245424 |
| RAD54B | RAD54 homolog B | 4.517781611 | 0.012218183 |
| EPCAM | epithelial cell adhesion molecule | 5.588964489 | 0.03276366 |
| RAD51AP1 | RAD51 associated protein 1 | 3.559518002 | 0.02738857 |
| STAG3 | stromal antigen 3 | 4.840431427 | 0.030929515 |
| CXCL2 | chemokine (C—X—C motif) | −5.230889636 | 0.030732912 |
| | ligan 2 | | |

1-23. (canceled)

24. A method of producing spermatogonial stem cells comprising,
co-culturing of THY1⁺/SSEA4⁻ cells isolated from a testicular tissue sample or their progeny with THY1⁻/SSEA4⁺ cells isolated from a testicular tissue sample; and
allowing colonies of the THY1⁻/SSEA4⁺ cells to form and expand.

25. The method of claim 24, wherein
the THY1⁺/SSEA4⁻ cells are grown as a monolayer on a substrate; and
the THY1⁻/SSEA4⁺ cells are grown on the monolayer of THY1⁺/SSEA4⁻ cells.

26. The method of claim 24, wherein
a suspension of THY1⁺/SSEA4⁻ cells is admixed with a suspension of THY1⁻/SSEA4⁺ cells and the resulting mixture is plated onto a substrate.

27. The method of claim 24, wherein
the isolated THY1⁺/SSEA4⁻ cells are treated to render them mitotically impaired prior to co-culture with the THY1−/SSEA4+ cells.

28. The method of claim 24, wherein
the THY1⁺/SSEA4⁻ cells and THY1⁻/SSEA4⁺ cells are derived from the same individual.

29. A method of instilling fertility in a male subject, comprising
obtaining a testicular tissue sample from the male subject;
digesting the testicular tissue sample to create a single cell suspension;
isolating THY1−/SSEA4+ cells from the single cell suspension;
co-culturing the THY1⁻/SSEA4⁺ cells with THY1⁺/SSEA4⁻ cells derived from a testicular tissue sample;
allowing colonies of the THY1⁻/SSEA4⁺ cells to form and expand;
harvesting the THY1⁻/SSEA4⁺ cells and cryopreserving them; and
transplanting the cryopreserved THY1⁻/SSEA4⁺ cells into the testes of the male subject.

30. The method of claim 29, wherein
the THY1⁺/SSEA4⁻ cells are grown as a monolayer on a substrate and the THY1⁻/SSEA4⁺ cells are grown on the monolayer of THY1⁺/SSEA4⁻ cells.

31. The method of claim 29, wherein
the male subject is a prepubertal male at the time that the testicular tissue sample is obtained.

32. The method of claim 29, wherein
the male subject is a cancer patient at the time the testicular tissue sample is obtained.

33. A method of isolating spermatogonial stem cells, comprising
obtaining a testicular tissue sample from a male subject;
digesting the testicular tissue sample to create a single cell suspension;
isolating THY1−/SSEA4+ cells from the single cell suspension.

34. The method of claim 33, wherein
the THY1−/SSEA4+ cells are isolated by fluorescence activated cell sorting.
